SEQUENCE LISTING (51 sequences)

SEQ ID NO: 1 - DNA, 1314 bp, Homo sapiens
atgctttggaaattgacggataatatcaagtacgaggactgcgaggaccgtcacgacggcaccagcaacgggacggcacggttgccccagctgggcactgtaggtcaatctccctacacgagcgccccgccgctgtcccacacccccaatgccgacttccagcccccatacttccccccaccctaccagcctatctacccccagtcgcaagatccttactcccacgtcaacgacccctacagcctgaaccccctgcacgcccagccgcagccgcagcacccaggctggcccggccagaggcagagccaggagtctgggctcctgcacacgcaccgggggctgcctcaccagctgtcgggcctggatcctcgcagggactacaggcggcacgaggacctcctgcacggcccacacgcgctcagctcaggactcggagacctctcgatccactccttacctcacgccatcgaggaggtcccgcatgtagaagacccgggtattaacatcccagatcaaactgtaattaagaaaggccccgtgtccctgtccaagtccaacagcaatgccgtctccgccatccctattaacaaggacaacctcttcggcggcgtggtgaaccccaacgaagtcttctgttcagttccgggtcgcctctcgctcctcagctccacctcgaagtacaaggtcacggtggcggaagtgcagcggcggctctcaccacccgagtgtctcaacgcgtcgctgctgggcggagtgctccggagggcgaagtctaaaaatggaggaagatctttaagagaaaaactggacaaaataggattaaatctgcctgcagggagacgtaaagctgccaacgttaccctgctcacatcactagtagagggagaagctgtccacctagccagggactttgggtacgtgtgcgaaaccgaatttcctgccaaagcagtagctgaatttctcaaccgacaacattccgatcccaatgagcaagtgacaagaaaaaacatgctcctggctacaaaacagatatgcaaagagttcaccgacctgctggctcaggaccgatctcccctggggaactcacggcccaaccccatcctggagcccggcatccagagctgcttgacccacttcaacctcatctcccacggcttcggcagccccgcggtgtgtgccgcggtcacggccctgcagaactatctcaccgaggccctcaaggccatggacaaaatgtacctcagcaacaaccccaacagccacacggacaacaacgccaaaagcagtgacaaagaggagaagcacagaaagtga

SEQ ID NO: 2 - DNA, 1296 bp, Homo sapiens
atgttagttcacagtttttcagccatggaccgtcacgacggcaccagcaacgggacggcacggttgccccagctgggcactgtaggtcaatctccctacacgagcgccccgccgctgtcccacacccccaatgccgacttccagcccccatacttccccccaccctaccagcctatctacccccagtcgcaagatccttactcccacgtcaacgacccctacagcctgaaccccctgcacgcccagccgcagccgcagcacccaggctggcccggccagaggcagagccaggagtctgggctcctgcacacgcaccgggggctgcctcaccagctgtcgggcctggatcctcgcagggactacaggcggcacgaggacctcctgcacggcccacacgcgctcagctcaggactcggagacctctcgatccactccttacctcacgccatcgaggaggtcccgcatgtagaagacccgggtattaacatcccagatcaaactgtaattaagaaaggccccgtgtccctgtccaagtccaacagcaatgccgtctccgccatccctattaacaaggacaacctcttcggcggcgtggtgaaccccaacgaagtcttctgttcagttccgggtcgcctctcgctcctcagctccacctcgaagtacaaggtcacggtggcggaagtgcagcggcggctctcaccacccgagtgtctcaacgcgtcgctgctgggcggagtgctccggagggcgaagtctaaaaatggaggaagatctttaagagaaaaactggacaaaataggattaaatctgcctgcagggagacgtaaagctgccaacgttaccctgctcacatcactagtagagggagaagctgtccacctagccagggactttgggtacgtgtgcgaaaccgaatttcctgccaaagcagtagctgaatttctcaaccgacaacattccgatcccaatgagcaagtgacaagaaaaaacatgctcctggctacaaaacagatatgcaaagagttcaccgacctgctggctcaggaccgatctcccctggggaactcacggcccaaccccatcctggagcccggcatccagagctgcttgacccacttcaacctcatctcccacggcttcggcagccccgcggtgtgtgccgcggtcacggccctgcagaactatctcaccgaggccctcaaggccatggacaaaatgtacctcagcaacaaccccaacagccacacggacaacaacgccaaaagcagtgacaaagaggagaagcacagaaagtga

SEQ ID NO: 3 - DNA, 1302 bp, Homo sapiens
atgtccatacttgccaaaatgggggactggcaggaccgtcacgacggcaccagcaacgggacggcacggttgccccagctgggcactgtaggtcaatctccctacacgagcgccccgccgctgtcccacacccccaatgccgacttccagcccccatacttccccccaccctaccagcctatctacccccagtcgcaagatccttactcccacgtcaacgacccctacagcctgaaccccctgcacgcccagccgcagccgcagcacccaggctggcccggccagaggcagagccaggagtctgggctcctgcacacgcaccgggggctgcctcaccagctgtcgggcctggatcctcgcagggactacaggcggcacgaggacctcctgcacggcccacacgcgctcagctcaggactcggagacctctcgatccactccttacctcacgccatcgaggaggtcccgcatgtagaagacccgggtattaacatcccagatcaaactgtaattaagaaaggccccgtgtccctgtccaagtccaacagcaatgccgtctccgccatccctattaacaaggacaacctcttcggcggcgtggtgaaccccaacgaagtcttctgttcagttccgggtcgcctctcgctcctcagctccacctcgaagtacaaggtcacggtggcggaagtgcagcggcggctctcaccacccgagtgtctcaacgcgtcgctgctgggcggagtgctccggagggcgaagtctaaaaatggaggaagatctttaagagaaaaactggacaaaataggattaaatctgcctgcagggagacgtaaagctgccaacgttaccctgctcacatcactagtagagggagaagctgtccacctagccagggactttgggtacgtgtgcgaaaccgaatttcctgccaaagcagtagctgaatttctcaaccgacaacattccgatcccaatgagcaagtgacaagaaaaaacatgctcctggctacaaaacagatatgcaaagagttcaccgacctgctggctcaggaccgatctcccctggggaactcacggcccaaccccatcctggagcccggcatccagagctgcttgacccacttcaacctcatctcccacggcttcggcagccccgcggtgtgtgccgcggtcacggccctgcagaactatctcaccgaggccctcaaggccatggacaaaatgtacctcagcaacaaccccaacagccacacggacaacaacgccaaaagcagtgacaaagaggagaagcacagaaagtga

SEQ ID NO: 4 - DNA, 1383 bp, Homo sapiens
atgcactcacctcctagagaccaggctgccatcatgctctggaagcttgtggagaatgtcaagtacgaagatatctatgaggaccggcacgatggtgtcccgagccacagctcgcggctctcccagctgggctcggtgtcccaaggaccctactcgagcgccccgccgctgtcccacaccccgtcgtcggacttccagccgccctacttcccacccccctaccagccgctcccctaccaccagagccaggacccctactcccacgtcaacgacccctactccctgaacccactgcaccagccccagcaacatccctgggggcaacggcagcggcaagaagtgggttcggaagccggctctctcctgccccagcctcgggccgccttgccccagctctcgggccttgacccccggagggactaccactcggtccgccggccggacgtgctgctgcattcggcgcaccacggcctggacgcgggcatgggtgacagcctctcgctgcacggcctcggccatcccggaatggaagacgtccagtcagttgaagatgccaataacagcggcatgaatctattggaccagtctgtcattaaaaaagttccagttcctcccaaatcggtgacttctctaatgatgaataaagacggcttcctgggaggcatgtctgtcaacaccggcgaggtgttttgctccgtcccaggccgtttgtctctgctcagttcaacttcgaagtacaaagtaactgtgggagaagttcagagacggctgtcgccccctgaatgcctcaatgcatctctcctcggcggagtcctcagaagagccaaatcgaaaaatggggggagatctttgcgagaaaggctagaaaaaatcggtttgaatttacccgcgggcaggcgcaaagcagcaaatgtcacgttactcacctccctggtggaaggagaagctgttcacttagctagggattttgggtacatttgcgaaacggagtttcccgccaaagccgtctctgagtatttgaaccggcagcacacagacccgagtgacctgcactcccgaaagaatatgctgttggccaccaagcaactttgtaaagaatttacggatctactggcgcaggaccggacaccgatagggaacagccgacccagccccatcctggagccggggatccagagctgcctcacgcacttcagcctcatcacgcacggcttcggcgccccggccatttgcgccgcgctcacggccctgcagaactatctcaccgaggcgctcaaaggcatggacaagatgttcttgaacaacaccaccactaacaggcacacgtctggggaaggcccaggtagtaaaactggcgacaaggaggagaaacacaggaaatga

SEQ ID NO: 5 - DNA, 2706 bp, Homo sapiens
atgttgtggaaaataaccgataatgtcaagtacgaagaggactgcgaggatcgccacgacgggagcagcaatgggaatccgcgggtcccccacctctcctccgccgggcagcacctctacagccccgcgccacccctctcccacactggagtcgccgaatatcagccgccaccctactttccccctccctaccagcagctggcctactcccagtcggccgacccctactcgcatctgggggaagcgtacgccgccgccatcaaccccctgcaccagccggcgcccacaggcagccagcagcaggcctggcccggccgccagagccaggagggagcggggctgccctcgcaccacgggcgcccggccggcctactgccccacctctccgggctggaggcgggcgcggtgagcgcccgcagggatgcctaccgccgctccgacctgctgctgccccacgcacacgccctggatgccgcgggcctggccgagaacctggggctccacgacatgcctcaccagatggacgaggtgcagaatgtcgacgaccagcacctgttgctgcacgatcagacagtcattcgcaaaggtcccatttccatgaccaagaaccctctgaacctcccctgtcagaaggagctggtgggggccgtaatgaaccccactgaggtcttctgctcagtccctggaagattgtcgctcctcagctctacgtctaaatacaaagtgacagtggctgaagtacagaggcgactgtccccacctgaatgcttaaatgcctcgttactgggaggtgttctcagaagagccaaatcgaaaaatggaggccggtccttgcgggagaagttggacaagattgggttgaatcttccggccgggaggcggaaagccgctcatgtgactctcctgacatccttagtagaaggtgaagctgttcatttggctagggactttgcctatgtctgtgaagccgaatttcctagtaaaccagtggcagaatatttaaccagacctcatcttggaggacgaaatgagatggcagctaggaagaacatgctattggcggcccagcaactgtgtaaagaattcacagaacttctcagccaagaccggacaccccatgggaccagcaggctcgccccagtcttggagacgaacatacagaactgcttgtctcatttcagcctgattacccacgggtttggcagccaggccatctgtgccgcggtgtctgccctgcagaactacatcaaagaagccctgattgtcatagacaaatcctacatgaaccctggagaccagagtccagctgattctaacaaaaccctggagaaaatggagaaacacaggaaataaatgttgtggaaaataaccgataatgtcaagtacgaagaggactgcgaggatcgccacgacgggagcagcaatgggaatccgcgggtcccccacctctcctccgccgggcagcacctctacagccccgcgccacccctctcccacactggagtcgccgaatatcagccgccaccctactttccccctccctaccagcagctggcctactcccagtcggccgacccctactcgcatctgggggaagcgtacgccgccgccatcaaccccctgcaccagccggcgcccacaggcagccagcagcaggcctggcccggccgccagagccaggagggagcggggctgccctcgcaccacgggcgcccggccggcctactgccccacctctccgggctggaggcgggcgcggtgagcgcccgcagggatgcctaccgccgctccgacctgctgctgccccacgcacacgccctggatgccgcgggcctggccgagaacctggggctccacgacatgcctcaccagatggacgaggtgcagaatgtcgacgaccagcacctgttgctgcacgatcagacagtcattcgcaaaggtcccatttccatgaccaagaaccctctgaacctcccctgtcagaaggagctggtgggggccgtaatgaaccccactgaggtcttctgctcagtccctggaagattgtcgctcctcagctctacgtctaaatacaaagtgacagtggctgaagtacagaggcgactgtccccacctgaatgcttaaatgcctcgttactgggaggtgttctcagaagagccaaatcgaaaaatggaggccggtccttgcgggagaagttggacaagattgggttgaatcttccggccgggaggcggaaagccgctcatgtgactctcctgacatccttagtagaaggtgaagctgttcatttggctagggactttgcctatgtctgtgaagccgaatttcctagtaaaccagtggcagaatatttaaccagacctcatcttggaggacgaaatgagatggcagctaggaagaacatgctattggcggcccagcaactgtgtaaagaattcacagaacttctcagccaagaccggacaccccatgggaccagcaggctcgccccagtcttggagacgaacatacagaactgcttgtctcatttcagcctgattacccacgggtttggcagccaggccatctgtgccgcggtgtctgccctgcagaactacatcaaagaagccctgattgtcatagacaaatcctacatgaaccctggagaccagagtccagctgattctaacaaaaccctggagaaaatggagaaacacaggaaataa

SEQ ID NO: 6 - DNA, 2040 bp, Homo sapiens
acaataaaatgtgtgcaaatactctccacagagataagtccccacctcagtcggtatagaaaagcatgataaaggttggaaaaaatggataaaaatgttgaaagaacctccagttccttattaggcgaagatacaatatttattccttgtctcctgggataaatctcttctgcctattgtcctcccagttctcagtgctgatgcttaattttcaaaacttctatattaccaagggacctacgacatcagccaaagaaatactcagcaagtacaaaatctgttccagggagctgcttttgtgcagagggaaaattttgttcctacaggtttttaagtgggtacgaggcaaggagctatctgatccgggcaaaaccattacaatttagatatctacctatagaacattttttttttcctttaaaaattggaaaatacaagaagtttggatttttttttcccgattctttttttggagggggaaattgcatcgtaagctttcggagaaacccaacatgtcaactacctttccgggactagtccacgatgccgagatacgtcacgacggatcaaacagctaccgtttgatgcagcttggctgtctggagtcagtagccaattccactgtcgcctattcctcctcctctcctttaacttactccaccaccggcaccgagtttgcgtccccctacttctccactaaccaccagtacaccccgctccaccaccagtccttccattacgagtttcagcacagccacccggccgtcacccccgacgcctactctctgaactctctccaccactcgcaacagtactaccagcagatccaccacggggagcccaccgactttattaacctgcacaatgcgcgggcgctcaagtcgtcctgcctggacgagcagaggcgggagctgggctgcctcgatgcctaccgccgccatgacctgtccctcatgagccatggctctcagtatggaatgcacccagatcaaagactcctgccagggcccagcctggggctggccgccgcgggagcagacgacttgcagggctctgtggaggcccagtgtgggcttgttctcaatggccaaggtggagtgataagaagaggtggcacctgtgtggtcaaccccacagacttattttgctctgtccctggccgtttgtcccttcttagttctacttccaaatacaaggtgaccattgctgaggtaaagaggcgcctctccccacctgagtgcctcaatgcttcactcttgggaggcattttgagaagagcaaaatcaaagaatgggggccgctgcctgagagagaaattggataggcttggcttaaacttaccagcaggaagacggaaagcagctaatgtcaccctccttacttccttggttgaaggggaggctttgcacttggctcgggattttggctacacttgtgaaacagagtttccagccaaagcagtaggagaacatcttgccagacaacatatggaacagaaagaacagacagcaagaaaaaagatgatcctggcgaccaaacaaatctgtaaagaattccaagacctcttgagccaagatagatcaccactgggatcctccagacccactccaattctagaccttgacatccagagacatttaacacatttcagtttgatcactcatggctttgggactccggcaatatgtgcagctctaagcactttccaaacagttctcagtgaaatgctgaactacttggaaaaacacactactcacaagaacggcggagcggcggattctggccaaggacatgccaactcggagaaagctcccctgcggaaaacttcagaggctgccgtgaaagagggcaaaacagaaaagacagactagctacatcaaacagaatctatttccagagagtcttgctgctgatattttttctaatatatatatcattgagggtgactaatcttcagtggaccaaatctctacccttccccaaccctccataaaaaaacaaaaatggaaatgaaaaatgaaacaaaaaaggacaaaacca

SEQ ID NO: 7 - DNA, 2245 bp, Homo sapiens
agaggaggaagggcgggcgctgggcacccgttgaccgacttttccaagtgcgatcagtgcccgtccgtcctgcctccatggacccgcccgggaacggccaccgctgaggaccccacgcccactaggatcccggctgggtcgcacccagctaccgcaccgtgacctccgcgggctgtgccggctcccggcgcctctgcccgcagcgctcgccgtcgggctagggctccgccgccgccacgcctcgcgcccggcactcaccgccccatgctggtgcacacctactccgccatggagcgccccgacgggctgggagcagctgccggcggggcccgcctgtcgtctctgccccaggcggcctacgggccggcgcccccgctctgccacacgccggccgccacagctgccgccgaattccagccgccctacttcccgccgccctacccgcagccaccgctgccctacggtcaggcgcccgacgccgccgcagcctttccccacctggcaggggacccatatggcggcctggcgcccctggcgcagccgcagcctcctcaggccgcctgggccgcgccccgcgcagccgcccgcgcccacgaggagcctcccggcctgctggcaccgcccgcccgcgccctgggccttgacccgcgccgtgactatgccactgccgtgccccggctcctgcacggcctggccgacggcgcgcacggcctggcagacgcacctctcggccttccggggctggcggcggcccccggtctggaggacctgcaggcaatggacgagccgggaatgagcctcctagaccagtccgtgatcaagaaagtgcccatcccctccaaagccagcagcctctcagccctctccttggccaaagacagcctggtgggcggcatcacaaatcctggtgaggtcttctgctccgtgcccggccggctttcactgctcagctcaacgtccaagtacaaggtgacggtgggggaggtgcagcggcgactctcgcctcccgagtgcctcaacgcctccctcctggggggtgtcctccgcagggccaagtccaaaaatgggggccggtgtttgcgggaacggttagagaagattgggctcaacctgccagctggccgtcgcaaggccgccaatgtgacgctgctgacttcgctagtggaaggagaggccgtgcacctggcccgagacttcggttacgtctgtgagacggagttcccagccaaggcagctgccgagtacctgtgccgacagcacgctgacccgggggagctgcacagccgcaagagcatgctgctggctgccaagcagatctgcaaggagtttgcagacttgatggctcaggaccgctcaccgctgggcaacagccgcccagcactcatcctggagcccggagtacagagctgcttgacacactttagcctcatcacccatggcttcggtgggcctgccatctgtgctgccctcactgccttccagaactatttgctggagtcactcaaggggctggacaagatgtttctaagcagtgtgggcagtgggcatggtgaaaccaaggcttcggagaaggatgccaagcatcggaaataactgcttctcccaccccatccctaaggggctcccaggccctgaaatagggacttagctcttgggggtgggcctggaaggactgaaaggtgggattagagtcaggccagaaagagaacattcatccagagatcccagagttggggatctggcttggagtaagggagggtggcctctctgtggtggtgtgttggtaagttaagggcccaggtatttgtctcatgtgtgcaattttctgacctttgatggttgagaagggtttggacagaaaattgacatgaaaagatctggctcatggggcagagccctttccattagcgtggctgggtggccgtgggtgcttctagaggccaaagcctttgtgtttttcactggtggcaggaggaaaattgataaatcagaggtgctactgaggagttggtgcccctcattccagaatctcctacccccagaaaaggggtgctggaaggaggccccagtggactctttgtacccttcctactctcagagagaagtgggcaggaggggtcctcaaggaacaaagaagataaagcacaaatcgcagaacttgaatccaggctgctgctcatcatagtcctgttgctgtctgtcctatttatttatgtatgttgtaattaaatttgaaattttaaaa

SEQ ID NO: 8 - DNA, 20 bp, Artificial Sequence
  Guide 1
ataccaggacgtgccgatgc

SEQ ID NO: 9 - DNA, 20 bp, Artificial Sequence
  Guide 2
cctccggcatcggcacgtcc

SEQ ID NO: 10 - DNA, 127 bp, Artificial Sequence
  Repair template
acacttttggcgatcaaggacagatacatccttataacaattgaatgccacagccactccacgcccttggggttgctcttttcctcgggcatcggcacgtcctggtattgctgaccacacacaaaag

SEQ ID NO: 11 - DNA, 22 bp, Artificial Sequence
  primer
ctcgatccactccttacctcac

SEQ ID NO: 12 - DNA, 21 bp, Artificial Sequence
  primer
attgctgttggacttggacag

SEQ ID NO: 13 - DNA, 22 bp, Artificial Sequence
  primer
taacagcggcatgaatctattg

SEQ ID NO: 14 - DNA, 22 bp, Artificial Sequence
  primer
caggaagccgtctttattcatc

SEQ ID NO: 15 - DNA, 22 bp, Artificial Sequence
  primer
gatcagacagtcattcgcaaag

SEQ ID NO: 16 - DNA, 22 bp, Artificial Sequence
  primer
gtagagctgaggagcgacaatc

SEQ ID NO: 17 - DNA, 18 bp, Artificial Sequence
  primer
tgctaaaggctggcacaa

SEQ ID NO: 18 - DNA, 20 bp, Artificial Sequence
  primer
taatcgctgtgcgtgtagtg

SEQ ID NO: 19 - DNA, 22 bp, Artificial Sequence
  primer
catgacctggaatcagatgaac

SEQ ID NO: 20 - DNA, 22 bp, Artificial Sequence
  primer
cgtgcgtgtgtattctgtattg

SEQ ID NO: 21 - DNA, 22 bp, Artificial Sequence
  primer
agtacaaaagtgtgcccaactg

SEQ ID NO: 22 - DNA, 22 bp, Artificial Sequence
  primer
tcgtttcagttgttcaaagcac

SEQ ID NO: 23 - DNA, 22 bp, Artificial Sequence
  primer
gtactgtcggtttcagaaatgc

SEQ ID NO: 24 - DNA, 22 bp, Artificial Sequence
  primer
attcagctggtcgatatcactg

SEQ ID NO: 25 - DNA, 20 bp, Artificial Sequence
  primer
ggtgcagtgaccaatcagaa

SEQ ID NO: 26 - DNA, 20 bp, Artificial Sequence
  primer
aatccgtcttcatccacagg

SEQ ID NO: 27 - DNA, 19 bp, Artificial Sequence
  primers
cctggtgtcatgggtttcc

SEQ ID NO: 28 - DNA, 19 bp, Artificial Sequence
  primer
gtcctgcagcacctgtctc

SEQ ID NO: 29 - DNA, 20 bp, Artificial Sequence
  primer
tgcccttcaacagattttgg

SEQ ID NO: 30 - DNA, 19 bp, Artificial Sequence
  primer
gcagcctttcattccttcc

SEQ ID NO: 31 - DNA, 23 bp, Artificial Sequence
  primer
tctcattcaacaagaaaccactg

SEQ ID NO: 32 - DNA, 21 bp, Artificial Sequence
  primer
ttcacgtctgtcacttccaca

SEQ ID NO: 33 - DNA, 20 bp, Artificial Sequence
  primer
acgatcagtccatcccagag

SEQ ID NO: 34 - DNA, 20 bp, Artificial Sequence
  primer
ttcacattgcacaaggcact

SEQ ID NO: 35 - DNA, 24 bp, Artificial Sequence
  primer
aacagagcaagatctgtatgttcg

SEQ ID NO: 36 - DNA, 22 bp, Artificial Sequence
  primer
ccacaacttttcttgtcacagc

SEQ ID NO: 37 - DNA, 22 bp, Artificial Sequence
  primer
ctttccctactgttcgttcacc

SEQ ID NO: 38 - DNA, 22 bp, Artificial Sequence
  primer
acgttatctgctggagctcttc

SEQ ID NO: 39 - DNA, 21 bp, Artificial Sequence
  primer
ccagccatgtatgtggctatc

SEQ ID NO: 40 - DNA, 22 bp, Artificial Sequence
  primer
cctcatagatggggacattgtg

SEQ ID NO: 41 - DNA, 22 bp, Artificial Sequence
  primer
tgatggaaaaggagttggactt

SEQ ID NO: 42 - DNA, 20 bp, Artificial Sequence
  primer
caggtccttggaaaccttga

SEQ ID NO: 43 - DNA, 20 bp, Artificial Sequence
  primer
cacggactctgtggagaacc

SEQ ID NO: 44 - DNA, 20 bp, Artificial Sequence
  primer
gcattcatagaggccatcgt

SEQ ID NO: 45 - protein, 437 aa, Homo sapiens
MLWKLTDNIKYEDCEDRHDGTSNGTARLPQLGTVGQSPYTSAPPLSHTPNADFQPPYFPPPYQPIYPQSQDPYSHVNDPYSLNPLHAQPQPQHPGWPGQRQSQESGLLHTHRGLPHQLSGLDPRRDYRRHEDLLHGPHALSSGLGDLSIHSLPHAIEEVPHVEDPGINIPDQTVIKKGPVSLSKSNSNAVSAIPINKDNLFGGVVNPNEVFCSVPGRLSLLSSTSKYKVTVAEVQRRLSPPECLNASLLGGVLRRAKSKNGGRSLREKLDKIGLNLPAGRRKAANVTLLTSLVEGEAVHLARDFGYVCETEFPAKAVAEFLNRQHSDPNEQVTRKNMLLATKQICKEFTDLLAQDRSPLGNSRPNPILEPGIQSCLTHFNLISHGFGSPAVCAAVTALQNYLTEALKAMDKMYLSNNPNSHTDNNAKSSDKEEKHRK

SEQ ID NO: 46 - protein, 431 aa, Homo sapiens
MLVHSFSAMDRHDGTSNGTARLPQLGTVGQSPYTSAPPLSHTPNADFQPPYFPPPYQPIYPQSQDPYSHVNDPYSLNPLHAQPQPQHPGWPGQRQSQESGLLHTHRGLPHQLSGLDPRRDYRRHEDLLHGPHALSSGLGDLSIHSLPHAIEEVPHVEDPGINIPDQTVIKKGPVSLSKSNSNAVSAIPINKDNLFGGVVNPNEVFCSVPGRLSLLSSTSKYKVTVAEVQRRLSPPECLNASLLGGVLRRAKSKNGGRSLREKLDKIGLNLPAGRRKAANVTLLTSLVEGEAVHLARDFGYVCETEFPAKAVAEFLNRQHSDPNEQVTRKNMLLATKQICKEFTDLLAQDRSPLGNSRPNPILEPGIQSCLTHFNLISHGFGSPAVCAAVTALQNYLTEALKAMDKMYLSNNPNSHTDNNAKSSDKEEKHRK

SEQ ID NO: 47 - protein, 433 aa, Homo sapiens
MSILAKMGDWQDRHDGTSNGTARLPQLGTVGQSPYTSAPPLSHTPNADFQPPYFPPPYQPIYPQSQDPYSHVNDPYSLNPLHAQPQPQHPGWPGQRQSQESGLLHTHRGLPHQLSGLDPRRDYRRHEDLLHGPHALSSGLGDLSIHSLPHAIEEVPHVEDPGINIPDQTVIKKGPVSLSKSNSNAVSAIPINKDNLFGGVVNPNEVFCSVPGRLSLLSSTSKYKVTVAEVQRRLSPPECLNASLLGGVLRRAKSKNGGRSLREKLDKIGLNLPAGRRKAANVTLLTSLVEGEAVHLARDFGYVCETEFPAKAVAEFLNRQHSDPNEQVTRKNMLLATKQICKEFTDLLAQDRSPLGNSRPNPILEPGIQSCLTHFNLISHGFGSPAVCAAVTALQNYLTEALKAMDKMYLSNNPNSHTDNNAKSSDKEEKHRK

SEQ ID NO: 48 - protein, 460 aa, Homo sapiens
MHSPPRDQAAIMLWKLVENVKYEDIYEDRHDGVPSHSSRLSQLGSVSQGPYSSAPPLSHTPSSDFQPPYFPPPYQPLPYHQSQDPYSHVNDPYSLNPLHQPQQHPWGQRQRQEVGSEAGSLLPQPRAALPQLSGLDPRRDYHSVRRPDVLLHSAHHGLDAGMGDSLSLHGLGHPGMEDVQSVEDANNSGMNLLDQSVIKKVPVPPKSVTSLMMNKDGFLGGMSVNTGEVFCSVPGRLSLLSSTSKYKVTVGEVQRRLSPPECLNASLLGGVLRRAKSKNGGRSLRERLEKIGLNLPAGRRKAANVTLLTSLVEGEAVHLARDFGYICETEFPAKAVSEYLNRQHTDPSDLHSRKNMLLATKQLCKEFTDLLAQDRTPIGNSRPSPILEPGIQSCLTHFSLITHGFGAPAICAALTALQNYLTEALKGMDKMFLNNTTTNRHTSGEGPGSKTGDKEEKHRK

SEQ ID NO: 49 - protein, 450 aa, Homo sapiens
MLWKITDNVKYEEDCEDRHDGSSNGNPRVPHLSSAGQHLYSPAPPLSHTGVAEYQPPPYFPPPYQQLAYSQSADPYSHLGEAYAAAINPLHQPAPTGSQQQAWPGRQSQEGAGLPSHHGRPAGLLPHLSGLEAGAVSARRDAYRRSDLLLPHAHALDAAGLAENLGLHDMPHQMDEVQNVDDQHLLLHDQTVIRKGPISMTKNPLNLPCQKELVGAVMNPTEVFCSVPGRLSLLSSTSKYKVTVAEVQRRLSPPECLNASLLGGVLRRAKSKNGGRSLREKLDKIGLNLPAGRRKAAHVTLLTSLVEGEAVHLARDFAYVCEAEFPSKPVAEYLTRPHLGGRNEMAARKNMLLAAQQLCKEFTELLSQDRTPHGTSRLAPVLETNIQNCLSHFSLITHGFGSQAICAAVSALQNYIKEALIVIDKSYMNPGDQSPADSNKTLEKMEKHRK

SEQ ID NO: 50 - protein, 452 aa, Homo sapiens
MSTTFPGLVHDAEIRHDGSNSYRLMQLGCLESVANSTVAYSSSSPLTYSTTGTEFASPYFSTNHQYTPLHHQSFHYEFQHSHPAVTPDAYSLNSLHHSQQYYQQIHHGEPTDFINLHNARALKSSCLDEQRRELGCLDAYRRHDLSLMSHGSQYGMHPDQRLLPGPSLGLAAAGADDLQGSVEAQCGLVLNGQGGVIRRGGTCVVNPTDLFCSVPGRLSLLSSTSKYKVTIAEVKRRLSPPECLNASLLGGILRRAKSKNGGRCLREKLDRLGLNLPAGRRKAANVTLLTSLVEGEALHLARDFGYTCETEFPAKAVGEHLARQHMEQKEQTARKKMILATKQICKEFQDLLSQDRSPLGSSRPTPILDLDIQRHLTHFSLITHGFGTPAICAALSTFQTVLSEMLNYLEKHTTHKNGGAADSGQGHANSEKAPLRKTSEAAVKEGKTEKTD

SEQ ID NO: 51 - protein, 442 aa, Homo sapiens
MLVHTYSAMERPDGLGAAAGGARLSSLPQAAYGPAPPLCHTPAATAAAEFQPPYFPPPYPQPPLPYGQAPDAAAAFPHLAGDPYGGLAPLAQPQPPQAAWAAPRAAARAHEEPPGLLAPPARALGLDPRRDYATAVPRLLHGLADGAHGLADAPLGLPGLAAAPGLEDLQAMDEPGMSLLDQSVIKKVPIPSKASSLSALSLAKDSLVGGITNPGEVFCSVPGRLSLLSSTSKYKVTVGEVQRRLSPPECLNASLLGGVLRRAKSKNGGRCLRERLEKIGLNLPAGRRKAANVTLLTSLVEGEAVHLARDFGYVCETEFPAKAAAEYLCRQHADPGELHSRKSMLLAAKQICKEFADLMAQDRSPLGNSRPALILEPGVQSCLTHFSLITHGFGGPAICAALTAFQNYLLESLKGLDKMFLSSVGSGHGETKASEKDAKHRK

(19) United States
(12) Patent Application Publication
(10) Pub. No.: US 2022/0088052 A1
(43) Pub. Date: Mar. 24, 2022

(54) TFAP2 INHIBITION FOR TREATING CARDIAC DISEASE INVOLVING FIBRO-FATTY REPLACEMENT

(71) Applicant: Koninklijke Nederlandse Akademie van Wetenschappen, Amsterdam (NL)

(72) Inventors: Eva van Rooij, Utrecht (NL); Arwa Obada Abdelrahman Reda Kohela, Utrecht (NL)

(73) Assignee: Koninklijke Nederlandse Akademie van Wetenschappen, Amsterdam (NL)

(21) Appl. No.: 17/424,505

(22) PCT Filed: Jan. 22, 2020
(86) PCT No.: PCT/EP2020/051489

§ 371 Date: Jul. 21, 2021

(30) Foreign Application Priority Data
Jan. 22, 2019 (EP) 19153135.9

(51) Int. Cl. A61K 31/713; A61K 31/165; A61K 31/055; A61K 31/609

(52) U.S. Cl. CPC A61K 31/713; A61K 31/609; A61K 31/055; A61K 31/165

(57) ABSTRACT

The present invention relates to novel treatments for treating cardiac disease involving fibro-fatty replacement, such as arrhythmogenic cardiomyopathy, atrial fibrillation, myocardial infarction and dilated cardiomyopathy. Such cardiac diseases can e.g. be caused by a mutation in a desmosomal protein such as plakophilin-2 (PKP2). The invention provides for agents for use in the prevention or treatment of such cardiac diseases, wherein the agent is at least one of: a) an agent that causes a reduction in expression in at least one TFAP2 subtype; and, b) an agent that causes a reduction in TFAP2-induced transcription. More preferably the agent is at least one of: a) an inhibitor of TFAP2; and, b) an agent that causes an increase in expression of PKP2.

### FIELD OF THE INVENTION

The invention relates to the field of medicine. In particular, it relates to the field of cardiology more specifically to the detrimental effects of fibro-fatty replacement during heart disease.

### BACKGROUND OF THE INVENTION

Arrhythmogenic cardiomyopathy (ACM) is a primary cardiomyopathy characterized by ventricular dysfunction due to a progressive dystrophy of the ventricular myocardium with fibro-fatty replacement and life-threatening ventricular arrhythmias in apparently healthy young people. The prevalence of the disease has been estimated at 1 in 5,000 individuals, although this estimate will likely increase as awareness of the condition increases among physicians. ACM is recognized as a cause of sudden death during athletic activity because of its association with ventricular arrhythmias that are provoked by exercise-induced catecholamine discharge (Gemayel et al. 2001).

ACM is an inherited disease of which the majority of mutations have been identified in genes encoding components of the desmosome, intercellular junctions that mechanically connect adjacent cells. Many of these mutations cause haploinsufficiency of the mutated desmosomal gene, meaning that the mutation causes loss of half of the level of functional desmosomal protein. This loss in protein is likely the cause for the disease. The most commonly mutated desmosomal protein in ACM is plakophilin-2 (PKP2) (Saffitz 2011). Mutations in proteins Desmoplakin (DSP), Desmoglein-2 (DSG2) Desmocollin-2 (DSC2) and Plakoglobin (JUP) have also been described as a cause for ACM (Corrado et al. 2017).

Fibro-fatty replacement of viable myocardium is a key feature of disease progression and loss of cardiac function during ACM. So far it was unclear how desmosomal protein mutations lead to the ACM phenotype and there are currently no etiology-specific cardioprotective treatments for ACM available. It is therefore that the far majority of patients with ACM are currently treated with angiotensin-converting enzyme (ACE) inhibitors, β-blockers and diuretics, similar to patients suffering from systolic heart failure. These drugs only involve suppression of ventricular arrhythmias, but they do not target the fibro-fatty remodeling process occurring during disease progression. Thus while these drugs manage help manage the disease, they do not reverse or block disease progression. Despite some improvements in therapy, there are currently no etiology-specific cardioprotective treatments for ACM, while the pathophysiology significantly differs from regular heart failure.

Fibro-fatty replacement has also been related to other primary cardiomyopathies such as atrial fibrillation, myocardial infarction and dilated cardiomyopathy. It is a goal of the current invention to provide for new and improved means and methods for treating cardiac disease wherein fibro-fatty replacement is part of the disease etiology.

### SUMMARY OF THE INVENTION

In a first aspect, the invention provides for an agent for use in the prevention or treatment of a cardiac disease wherein fibro-fatty replacement is part of the disease etiology, wherein the agent is at least one of a) an agent that causes a reduction in expression in at least one TFAP2 subtype and, b) an agent that causes a reduction in TFAP2-induced transcription.

In a second aspect, the invention provides for a composition comprising an agent for reducing the expression of TFAP2 as defined herein and a pharmaceutically acceptable excipient for a use in the prevention or treatment of a cardiac disease wherein fibro-fatty replacement is part of the disease etiology.

In a third aspect, the invention provides for an in vivo, in vitro, or ex vivo method for reducing TFAP2 expression, the method comprising the step of contacting a cell with an agent as defined herein or with a composition as defined herein.

In a fourth aspect, the invention provides for a method for reducing TFAP2 expression in a subject in need thereof, the method comprising the step of administering an effective amount of an agent as defined in herein, or a composition as defined herein.

In a fifth aspect, the invention provides for a method for identifying an agent that causes at least one of a reduction in TFAP2 expression, and a reduction in TFAP2-induced transcription, the method comprising the steps of:

a) contacting a PKP2-deficient epicardial cell with a candidate agent;

b) determining in the cell in a) at least one of the level of TFAP2 expression and the level of TFAP2-induced transcription;

c) identifying the agent as an agent that causes a reduction in TFAP2 expression if the level of TFAP2 expression as in determined in b) is less that the level in a corresponding control cell in the absence of the agent, and,

identifying the agent as an agent that causes a reduction in TFAP2-induced transcription if the level of TFAP2 expression as in determined in b) is less that the level in a corresponding control cell in the absence of the agent.

In a sixth aspect, the invention provides for an apparatus comprising: an implantable source; and an implantable catheter for delivering the agent according to claims 1-9 from the source to the pericardial sac region of a heart in a patient with a cardiac disease.

In a seventh aspect, the invention provides for a gene therapy vector comprising the agent as defined herein, wherein preferably the gene therapy vector is an adeno-associated viral vector (AAV).

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method

For purposes of the present invention, the following terms are defined below.

The terms “AP2” and “TFAP2” to are both used interchangeable herein as abbreviations for the Transcription Factor Activating Enhancer-Binding Protein 2 family of genes.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of a cancer varies depending upon the manner of administration, the age, body weight, and general health of the subject. Ultimately, the attending physician or veterinarian will decide the appropriate amount and dosage regimen.

Such amount is referred to as an “effective” amount. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use. Agents that causes a reduction in expression in at least one TFAP2 subtype or agents that causes a reduction in TFAP2-induced transcription or compositions according to this invention are preferably for use in methods or uses according to this invention.

Throughout this application, expression is considered to be the transcription of a gene into functional mRNA, leading to a polypeptide such as an enzyme or transcription factor. A polypeptide can assert an effect or have an activity. In this context, increased or decreased expression of a polypeptide can be considered an increased or decreased level of mRNA encoding said polypeptide, an increased or decreased level or amount of polypeptide molecules, or an increased or decreased total activity of said polypeptide molecules. Preferably, an increased or decreased expression of a polypeptide results in an increased or decreased activity of said polypeptide, respectively, which can be caused by increased or decreased levels or amounts of polypeptide molecules. More preferably, a reduction of TFAP2 expression is a reduction of transcription of a TFAP2 gene, destabilization or degradation of TFAP2 mRNA, reduction in TFAP2-induced transcription reduction of the amount of TFAP2 polypeptide molecules, reduction of TFAP2 polypeptides molecule activity, destabilization or degradation of TFAP2 polypeptide, or combinations thereof. A destabilized mRNA leads to lower expression of its encoded polypeptide, possibly it cannot lead to such expression. A degraded mRNA is destroyed and cannot lead to expression of its encoded polypeptide. A destabilized polypeptide asserts less of an effect or has lower activity than the same polypeptide that has not been destabilized, possibly it asserts no effect or has no activity. A destabilized polypeptide can be denatured or misfolded. A degraded polypeptide is destroyed and does not assert an effect or have an activity.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

By “pluripotency” and pluripotent stem cells it is meant that such cells have the ability to differentiate into all types of cells in an organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to alkaline phosphatase, SSEA3, SSEA4, Sox2, Oct3/4, Nanog, TRA160, TRA181 TDGF 1 Dnmt3b FoxD3 GDF3 Cyp26a1 TERT and zfp42. In addition, the iPS cells are capable of forming teratomas. In addition, they are capable of forming or contributing to ectoderm, mesoderm, or endoderm tissues in a living organism.

Any reference to nucleotide or amino acid sequences accessible in public sequence databases herein refers to the version of the sequence entry as available on the filing date of this document.

### DETAILED DESCRIPTION OF THE INVENTION

The present inventors have surprisingly found that Transcription Factor Activating Enhancer-Binding Protein 2 (AP2 or TFAP2) is responsible for the fibro-fatty replacement that is the cause of the loss of function in several cardiac diseases. Furthermore, the inventors show that reduction of the expression of at least one TFAP2 subtype or reduction in TFAP2 induced transcription blocks the conversion of epicardial cells into fibro-fatty cells and is therefore thus a relevant target for the treatment of cardiac diseases where in fibro-fatty replacement is part of the disease etiology.

Accordingly, in a first aspect, the present invention provides for an agent for use in the prevention or treatment of a cardiac disease wherein fibro-fatty replacement is part of the disease etiology, wherein the agent is at least one of a) an agent that causes a reduction in expression in at least one TFAP2 subtype and b) an agent that causes a reduction in TFAP2-induced transcription.

The TFAP2 (AP-2) family is a family of basic helix-span-helix transcription factors which have been recognized to play key roles during development. The TFAP2 family consist of five closely related family members:
TFAP2A (consisting of variant 1 (SEQ ID NO: 45 Genbank accession number: NP_003211.1), variant 2 (SEQ ID NO: 46 Genbank accession number: NP_001027451.1) and consisting of variant 3 (SEQ ID NO: 47 Genbank accession number: NP_001035890.1);
TFAP2B, (SEQ ID NO: 48 Genbank accession number: NP_003212.2);
TFAP2C, (SEQ ID NO: 49 Genbank accession number: NP_003213.1);
TFAP2D (SEQ ID NO: 50 Genbank accession number: NP_758438.2); and
TFAP2E (SEQ ID NO: 51 Genbank accession number: NP_848643.2);
   The family members are thought to thought to form homo- and heterodimers that bind to similar promoter sequences albeit with different affinities. The inventors found that expression of all five family members (subtypes) was increased in cells having a mutation in genes encoding components of the desmosome such as plakophilin-2 (PKP2). Following this observation, the inventors have shown that reducing the expression of at least one subtype of TFAP2 blocks the conversion of epicardial cells into fibro-fatty cells. Likewise, the reduction of TFAP2 induced transcription was also found to blocks the conversion of epicardial cells into fibro-fatty cells.

Accordingly in one embodiment, an agent for use according to the invention reduces the expression of at least one of TFAP2A, TFAP2B, TFAP2C, TFAP2D and TFAP2E. In one embodiment, the agent or a combination of at least two agents according to the invention reduces the expression of at least TFAP2A, TFAP2B and TFAP2C. Preferably, the agent or a combination of at least two agents according to the invention reduces the expression of each of TFAP2A, TFAP2B TFAP2C, TFAP2D and TFAP2E. According to one embodiment, an agent for use according to the invention, alternatively or in addition, causes a reduction in TFAP2-induced transcription. The agent preferably reduces transcription of at least one gene comprising a TFAP2 family member consensus binding site as e.g. defined by Scott et al. (eLife 2018; 7:e36330. DOI: https://doi.org/10.7554/eLife.36330). The agent preferably reduces transcription of at least one of a fat marker and a fibroblast marker as herein defined below. Preferably, the agent preferably reduces transcription of the fat marker and/or fibroblast marker in cells of the epicardium.

In a preferred embodiment, the agent that causes an increase in expression of PKP2 is an inhibitor of Wnt3a. Inhibitors of Wnt3a are known in the art (see e.g. Scott et al., 2017, supra) and include e.g. Tricostatin A, hexachlorophene, niclosamide and anti-Wnt3a antibodies. An example of an anti-Wnt3a antibody is the rabbit monoclonal (IgG) antibody clone 1 H12L14 (Thermo Fisher, USA) and the mouse monoclonal (IgG2a) ab81614 (clone 3A6) (Abcam, United Kingdom).

In one embodiment, the agent for a use according the invention is at least one of an inhibitor of TFAP2 and an agent that causes an increase in expression of a desmosomal protein. Preferably, the agent causes the increase of a desmosomal protein preferably selected from the group consisting of plakophilin-2 (PKP2), Desmoplakin (encoded by DSP), Desmoglein-2 (DSG2) Desmocollin-2 (DSC2) and Plakoglobin (JUP). More preferably, the agent at least causes an increase of expression of at least one of protein plakophilin-2 (PKP2) and plakoglobin (JUP).

Plakophilin-2 (PKP2) may have the reference amino acid sequence of NP_001005242.2 (isoform 2a) or NP_004563.2 (isoform 2b) and may be encoded by the reference nucleotide sequence of NM_001005242.2 (isoform 2a) or NM_004572.3 (isoform 2b).

Desmoplakin (DSP) may have the reference amino acid sequence of NP_004406.2 (isoform I), NP_001305963.1 (isoform Ia) or NP_001008844.1 (isoform II) and may be encoded by the reference nucleotide sequence of NM_004415.4 (isoform I), NM_001319034.1 (isoform Ia) or NM_001008844.2 (isoform II).

Desmoglein-2 (DSG2) may have the reference amino acid sequence of XP_024306863.1 and may be encoded by the reference nucleotide sequence of XM_024451095.1.

Desmocollin-2 (DSC2) may have the reference amino acid sequence of XP_005258263.1 and may be encoded by the reference nucleotide sequence of XM_005258206.4.

Plakoglobin (JUP) may have the reference amino acid sequence of NP_001339702 and may be encoded by the reference nucleotide sequence of NM_001352773.

In one embodiment the agent for a use according to the invention, is (a source of) at least one of a genome editing complex, an antibody, a compound. Preferably the agent is a nucleic acid molecule such as a siRNA or miRNA. More preferably, the agent is at least one of: a genome editing complex that restores a deficiency in a desmosomal protein, preferably as defined above or inactivates TFAP2, a neutralizing antibody against TFAP2 and a siRNA complementary to TFAP2 mRNA. The source of the genome editing complex, antibody or nucleic acid molecule can e.g. be a gene therapy vector encoding the genome editing molecule, a gene therapy vector encoding a desmosomal protein, preferably as defined above, an antibody or a nucleic acid molecule. Gene therapy vectors are well-known in the art and include e.g. adenovirus associated virus (AAV)-based vector as further described herein.

In one embodiment, the agent for a use according to the invention is a genome editing molecule that restores PKP2 deficiency or inactivates TFAP2. The genome editing complex can be any entity that modulates or alters the genome. The genome editing complex can be for example, a CRISP-CAS molecule such as CRISPR-Cas-9, CRISPR-Cas-12a, or CRISPR-Cas-13 with a guide RNA that directs restoration of a PKP2 deficiency or that directs inactivation of at least one TFAP2.

In one embodiment, the agent for a use according to the invention is an antibody that a causes the reduction in expression in at least one TFAP2 subtype or an antibody that causes a reduction in TFAP2-induced transcription.

In one embodiment, the agent for a use according to the invention is a compound that a causes the reduction in expression in at least one TFAP2 subtype or a compound that causes a reduction in TFAP2-induced transcription.

In one preferred embodiment, the agent for a use according to the invention is a nucleic acid molecule such as an siRNA, and miRNA or an antisense oligonucleotide (AON). Preferably the agent for a use according to the invention is an siRNA that is complementary at least one of TFAP2A variant 1 (SEQ ID NO:1; reference sequence NM_003220.3), TFAP2A variant 2 (SEQ ID NO:2; reference sequence NM_001032280.2), TFAP2A variant 3 (SEQ ID NO:3; reference sequence NM 001042425.1), TFAB2B (SEQ ID NO:4; reference sequence NM_003221.4), TFAP2C (SEQ ID NO: 5; reference sequence NM_003222.4), TFAP2D (SEQ ID NO:6, reference sequence NM_172238) and TFAP2E (SEQ ID NO:7, reference sequence NM_178548.4) More preferably, the agent for a use according to the invention is an siRNA is represented by Origene (Rockville, Md. 20850, USA, www.origene.com) catalogue numbers # (SR304787), (SR304788), (SR304789) and (SR303544).

In one embodiment, the agent for a use in accordance with the invention is an agent, the administration of which is causes an increase or prevents the loss of the expression of an epicardial marker, preferably in cells of the epicardium. More preferably, the administration of the agent causes an increase or prevents the loss of the expression of WT1. The increase in expression of WT1 or the loss of expression of WT1 can be determined by techniques commonly known in the art. For example, the reduction of loss or the increase of the expression of WT1 be determined using PCR techniques such as qPCR, western blot or flow cytometry (see also FIGS. 2 and 3). In this context, a reduction of loss or increase is preferably a reduction of loss or an increase of expression as compared to either a predetermined value, or to a reference value. A preferred reference value is a reference value obtained by determining WT1 expression levels in sample obtained from individuals that do not suffer from a cardiac disease wherein fibro-fatty replacement is part of the disease etiology. A reduction of loss of expression of WT1 levels preferably means that the loss of expression is reduced by at least 10, 15, 20, 25, 30, 35, 40, 45, 50, 55, 60, 65, 70, 75, 80, 85, 90, 95, 96, 97, 98, 99, or 100%. An increase in the expression of WT1 preferably means that the expression is increased by at least 10, 15, 20, 25, 30, 35, 40, 45, 50, 55, 60, 65, 70, 75, 80, 85, 90, 95, 96, 97, 98, 99, or 100%.

In one embodiment, the agent for a use in accordance with the invention is an agent, the administration of which is causes inhibition or suppression of the expression of at least one of a fat marker and a fibroblast marker, preferably in cells of the epicardium. More preferably, the administration of the agent causes inhibition or suppression of the expression of a fat marker selected from the group consisting of CEBPA, EBF3, RORA, PPARG, PPARGC1A and UCP1; and the administration of the agent causes inhibition or suppression of the expression a fibroblast marker selected from the group consisting of COL1A2, COL2A1, POSTN, FN1 and ACTA2.

The inhibition or suppression of the fat-markers CEBPA, EBF3, RORA, PPARG, PPARGC1A and/or UCP1 or fibroblast markers COL1A2, COL2A1, POSTN, FN1 and/or ACTA2, can be measured by any method commonly known in the art. For example, the inhibition or suppression can be determined by using PCT-techniques, such as qPCR (using e.g. the primers in Table 2), western blot, flow cytometry or immunofluorescent staining (see for example FIG. 2). In this context, inhibition or suppression is compared to either a predetermined value, or to a reference value. A preferred reference value is a reference value obtained by determining the expression levels of the fat-markers or of the fibroblast markers in an untreated sample containing cells or cell extracts. This untreated sample can be from the same subject or from a different and healthy subject, more preferably it is a sample that was obtained in the same way, thus containing the same type of cells. Alternately, the test sample was obtained from the subject before treatment commenced. Inhibition or suppression preferably means that the expression of the fat markers or fibroblast markers is reduced by at least 10, 15, 20, 25, 30, 35, 40, 45, 50, 55, 60, 65, 70, 75, 80, 85, 90, 95, 96, 97, 98, 99, or 100%.

In one embodiment, the agent for a use according to the invention is administered to a subject intermittently or continuously, preferably continuously. The effective amount of an agent according to the invention, is dictated by and directly dependent on the unique characteristics of the active compound and the particular therapeutic effect to be achieved, and the limitations inherent in the art of compounding such an active compound for the treatment of individuals.

Generally an effective administered amount of an agent according to the invention will depend on the relative efficacy of the compound chosen, the half-life of the compound and the method of administration.

In one embodiment, the invention provides for an agent for a use described herein, wherein the agent is administered locally to the epicardium/pericardial sac region. In other words, the agent according to the invention can be administered to the surface of the epicardium or within the pericardial sac. An example of epicardial administration would be pericardiocentesis. Preferably, the site of epicardial administration is on the left ventricle. Pericardiocentesis is herein defined as administration into the pericardial space. Pericardiocentesis can be achieved by direct injection using a needle which is inserted through the pericardium into the pericardial space (space between the pericardium and epicardium). Often pericardiocentesis is used in conjunction with imaging techniques such as fluoroscopy or ultrasound.

In an embodiment the agent for a use according to the invention reduces TFAP2 expression or TFAP2-induced transcription. This TFAP2 expression or TFAP2-induced transcription is preferably the overall TFAP2 expression or TFAP2-induced transcription of the subject that is treated. The level of TFAP2 expression or TFAP2-induced transcription can be determined using methods known in the art, or exemplified in the examples. For example, TFAP2 expression or TFAP2-induced transcription can be determined using PCR techniques such as RT-PCR, or using immunostaining, mass spectrometry, western blot or ELISA, for example on a sample containing cells or cell extracts, preferably obtained from the subject. In this context, a reduction is preferably a reduction as compared to either a predetermined value, or to a reference value. A preferred reference value is a reference value obtained by determining TFAP2 expression levels or TFAP2-induced transcription levels in an untreated sample containing cells or cell extracts. This untreated sample can be from the same subject or from a different and healthy subject, more preferably it is a sample that was obtained in the same way, thus containing the same type of cells. Conveniently, both the test sample and the reference sample can be part of a single larger sample that was obtained. Alternately, the test sample was obtained from the subject before treatment commenced. A highly preferred reference value is the expression level of TFAP2 or TFAP2-induced transcription level in a sample obtained from a subject prior to the first administration of an agent for use according to the invention. A reduction of expression of at least one TFAP2 subtype preferably means that expression is reduced by at least 10, 15, 20, 25, 30, 35, 40, 45, 50, 55, 60, 65, 70, 75, 80, 85, 90, 95, 96, 97, 98, 99, or 100%. If expression of at least one TFAP2 subtype is reduced by for example 100%, it may be that expression of a TFAP2 subtype can no longer be detected. Reduction can be assessed at the protein level, for example through immunostaining, western blotting, ELISA, or mass spectrometry, or it can be assessed at the mRNA level, for example through PCR techniques such as RT-PCR. In preferred embodiments, the invention provides an agent that causes a reduction in expression in at least one TFAP2 subtype for use according to the invention, wherein the reduction of TFAP2 expression is determined using PCR, western blot or immunostaining, wherein a preferred PCR technique is RT-PCR. In preferred embodiments the invention provides an agent that causes a reduction in expression in at least one TFAP2 subtype for use according to the invention, wherein TFAP2 expression is reduced by at least 10%, 20%, 30%, 40%, 50%, 60%, 70%, 80%, 90%, 95% or more. In the most preferred embodiments, TFAP2 expression is reduced by about 100%, preferably by 100%.

A reduction of expression TFAP2-induced transcription level preferably means that the transcription level expression is reduced by at least 10, 15, 20, 25, 30, 35, 40, 45, 50, 55, 60, 65, 70, 75, 80, 85, 90, 95, 96, 97, 98, 99, or 100%. If expression of TFAP2-induced transcription level is reduced by for example 100%, it may be that expression of a TFAP2-induced transcription level can no longer be detected. Reduction can be assessed at the protein level, for example through immunostaining, western blotting, ELISA, or mass spectrometry, or it can be assessed at the mRNA level, for example through PCR techniques such as RT-PCR. In preferred embodiments, the invention provides an agent that causes a reduction in expression in TFAP2-induced transcription level for use according to the invention, wherein the reduction of TFAP2-induced transcription is determined using PCR, western blot or immunostaining, wherein a preferred PCR technique is RT-PCR. In preferred embodiments the invention provides an agent that causes a reduction in expression in TFAP2-induced transcription level for use according to the invention, wherein TFAP2-induced transcription expression is reduced by at least 10%, 20%, 30%, 40%, 50%, 60%, 70%, 80%, 90%, 95% or more. In the most preferred embodiments, TFAP2-induced transcription level is reduced by about 100%, preferably by 100%.

In an embodiment, the invention provides for an agent for use according to the invention, wherein the cardiac disease wherein fibro-fatty replacement is part of the disease etiology, is at least one of arrhythmogenic cardiomyopathy, atrial fibrillation, myocardial infarction and dilated cardiomyopathy. These diseases are characterized by the fact that the viable myocardium located at the epicardial surface of the heart is increasingly replaced by fibro-fatty areas. The disease progression is further characterized by a loss of the expression of the epicardial marker WT1 and an increase in the expression the fat-markers CEBPA, EBF3, RORA, PPARG, PPARGC1A and/or UCP1 or fibroblast markers COL1A2, COL2A1, POSTN, FN1 and/or ACTA2.

In an embodiment, the agent according to the invention is for use in the treatment of a cardiac disease wherein fibro-fatty replacement is part of the disease etiology that is caused by a mutation in a desmosomal protein. Preferably, the mutation is in and a desmosomal protein selected from the group consisting of Desmoplakin (DSP), Desmoglein-2 (DSG2) Desmocollin-2 (DSC2) and Plakoglobin (JUP). More preferably the mutated desmosomal protein is plakophilin-2 (PKP2). Even more preferably, the mutated desmosomal protein is PKP2 and the mutation is c.2013delC.

In a further aspect, the invention provides a composition comprising an agent for reducing the expression of TFAP2 as defined herein and a pharmaceutically acceptable excipient, for use in the prevention or treatment of a cardiac disease wherein fibro-fatty replacement is part of the disease etiology. Such a composition is referred to herein as a composition for use according to the invention. Preferred compositions for use according to the invention are pharmaceutical compositions. In preferred embodiments, the pharmaceutical composition for use according to the invention is formulated for oral, sublingual, parenteral, intravascular, intravenous, subcutaneous, or transdermal administration. Preferably the pharmaceutical composition for use according to the invention is administered to a subject intermittently or continuously. More preferably, the pharmaceutical composition is administered continuously.

The compositions may be administered alone or in combination with other pharmaceutical or cosmetic agents and can be combined with a physiologically acceptable carrier thereof. In particular, the compounds described herein can be formulated as pharmaceutical or cosmetic compositions by formulation with additives such as pharmaceutically or physiologically acceptable excipients carriers, and vehicles. Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003), 21st edition (2005) and 22nd edition (2012), incorporated herein by reference.

Compositions for use according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in a conventional manner using one or more physiologically acceptable carriers comprising excipients and auxiliaries that facilitate processing of the active compounds into preparations which can be used pharmaceutically. Proper formulation is dependent on the route of administration chosen.

For example, the agents and compositions for use according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents.

Compositions for parenteral administration include aqueous solutions of the compositions in water soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

One aspect of the invention provides an in vivo, in vitro, or ex vivo method for reducing TFAP2 expression or TFAP2-induced transcription, the method comprising the step of contacting a cell with an agent as defined herein, or with a pharmaceutical composition as defined herein. Preferably, the method is for treating a cardiac disease wherein fibro-fatty replacement is part of the disease etiology, such as arrhythmogenic cardiomyopathy, atrial fibrillation, myocardial infarction and dilated cardiomyopathy. Preferred methods comprise contacting a cell with a composition comprising an agent that reduces TFAP2 expression or TFAP2-induced transcription as defined earlier herein. In the context of the invention, contacting a cell with an agent that reduces TFAP2 expression or TFAP2-induced transcription or a composition comprising an agent that reduces TFAP2 expression or TFAP2-induced transcription can comprise adding such agent or composition to a medium in which a cell is cultured. Contacting a cell with an agent that reduces TFAP2 expression or TFAP2-induced transcription a composition can also comprise adding such agent or composition to a medium, buffer, or solution in which a cell is suspended, or which covers a cell. Other preferred methods of contacting a cell comprise injecting a cell with an agent that reduces TFAP2 expression or TFAP2-induced transcription or composition, or exposing a cell to a material comprising an agent that reduces TFAP2 expression or TFAP2-induced transcription. Preferably, the cell is an epicardial cell.

In one embodiment of this aspect, the method is an in vitro method. In a further embodiment of this aspect, the method is an ex vivo method. In a further embodiment of this aspect, the method is an in vivo method. In a preferred embodiment of this aspect, the method is an in vitro or an ex vivo method. Within the embodiments of this aspect, the cell may be a cell from a sample obtained from a subject. Such a sample may be a sample that has been previously obtained from a subject. Within the embodiments of this aspect, samples may have been previously obtained from a human subject. Within the embodiments of this aspect, samples may have been obtained from a non-human subject. In a preferred embodiment of this aspect, obtaining the sample is not part of the method according to the invention.

In an embodiment, the method according to the invention is a method for reducing TFAP2 expression in a subject in need thereof the method comprising the step of administering an effective amount of an agent as defined herein, or a composition defined herein. Preferably, the method is for the treatment of a cardiac disease wherein fibro-fatty replacement is part of the disease etiology, preferably the cardiac disease is arrhythmogenic cardiomyopathy, atrial fibrillation, myocardial infarction and dilated cardiomyopathy. More preferably, the disease is arrhythmogenic cardiomyopathy.

In a further aspect, the invention provides for a method for identifying an agent that causes at least one of a reduction in TFAP2 expression, and a reduction in TFAP2-induced transcription, the method comprising the steps of: a) contacting a PKP2-deficient epicardial cell with a candidate agent; b) determining in the cell in a) at least one of the level of TFAP2 expression and the level of TFAP2-induced transcription; c) identifying the agent as an agent that causes a reduction in TFAP2 expression if the level of TFAP2 expression as in determined in b) is less that the level in a corresponding control cell in the absence of the agent, and, d) identifying the agent as an agent that causes a reduction in TFAP2-induced transcription if the level of TFAP2 expression as in determined in b) is less that the level in a corresponding control cell in the absence of the agent. The method preferably is an an in vitro or an ex vivo method.

In another aspect, the invention provides for an apparatus for delivering an agent of the invention. Preferably the apparatus is for delivering the agent to the pericardial sac region of a heart in a patient to be treated in accordance with the invention. The apparatus comprises a source of the agent to be delivered and a catheter for delivering the agent from the source to the pericardial sac region. Preferable at least the catheter is an implantable catheter. Optionally both the source and the catheter are implantable. The source can be or comprises a pump that delivers the agent under pressure to the catheter, or can an access port into which the agent is injected using a syringe and thereby delivering the agent by the pressure exerted through the plunger of the syringe. The catheter has its proximal end connected to source and its distal end inserted into pericardial sac. Preferably the agent of the invention to be delivered by the apparatus is formulated as a liquid formulation. An apparatus according to the invention has for example been described in WO2006122140.

An agent according to the invention may be indirectly administrated using suitable means known in the art. It may for example be provided to an individual or a cell, tissue or organ of said individual as such. It may also be administered in the form of an expression vector wherein the expression vector encodes an RNA transcript comprising the sequence of an agent according to the invention. The expression vector is preferably introduced into a cell, tissue, organ or individual via a gene therapy vector. In a preferred embodiment, there is provided a viral-based expression vector comprising an expression cassette or a transcription cassette that drives expression or transcription of an agent according to the invention.

A cell can be provided with an agent according to the invention by viral expression provided by adenovirus- or adeno-associated virus-based vectors. Expression may be driven by an RNA polymerase II promoter (Pol II) such as a U7 RNA promoter or an RNA polymerase III (Pol III) promoter, such as a U6 RNA promoter. A preferred delivery vehicle is a viral vector such as an adeno-associated virus vector (AAV), or a retroviral vector such as a lentivirus vector and the like. Also, plasmids, artificial chromosomes, plasmids usable for targeted homologous recombination and integration in the human genome of cells may be suitably applied for delivery of an agent according to the invention. Preferred for the invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol-III driven transcripts, preferably, in the form of a fusion transcript with an U1 or U7 transcript. Such fusions may be generated as previously described (Gorman et al., 1998).

A preferred expression system for an agent according to the invention is an adenovirus associated virus (AAV)-based vector. Single chain and double chain AAV-based vectors have been developed that can be used for prolonged expression of nucleotide sequences for highly efficient gene therapy. Accordingly, the invention provides for a gene therapy vector comprising the agent according the invention, wherein the gene therapy vector preferably is an adeno-associated viral vector (AAV).

A preferred AAV-based vector, for instance, comprises an expression cassette that is driven by an RNA polymerase III-promoter (Pol III) or an RNA polymerase II promoter (Pol II). A preferred RNA promoter is, for example, a Pol III U6 RNA promoter, or a Pol II U7 RNA promoter.

The invention accordingly provides for a viral-based vector, comprising a Pol II or a Pol III promoter driven expression cassette for expression of an agent according to the invention.

An AAV vector according to the invention is a recombinant AAV vector and refers to an AAV vector comprising part of an AAV genome comprising an encoded agent according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV8, AAV9 and others. A protein shell comprised of capsid protein may be derived from an AAV serotype such as AAV1, 2, 3, 4, 5, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention.

All patent and literature references cited in the present specification are hereby incorporated by reference in their entirety.

The present invention is further described by the following examples, which should not be construed as limiting the scope of the invention.

### DESCRIPTION OF THE FIGURES

FIG. 1: Differentiation of hiPSCs into epicardial cells. (A) Schematic of CRISPR-Cas9-mediated targeting strategy to revert PKP2 c.2013delC mutation. A guide RNA and DNA repair template containing the WT PKP2 sequence were designed to target the PKP2 locus. Blocking mutations were introduced to prevent target re-cutting. The middle panel shows sequencing results in mutant and reverted cells. Mutation site correction is indicated by an arrow. PKP2 c.2013delC and reverted hiPSCs were subsequently differentiated into epicardial cells. (B) Schematic of hiPSC to epicardial cell differentiation protocol using WNT pathway modulators. (C,D) Analysis of WT1 protein expression by (C) Western blot and (D) flow cytometry in PKP2 c.2013delC and reverted hiPSC-epicardial cells. (E) Immunofluorescent staining of epicardial markers in PKP2 c.2013delC and reverted hiPSCs differentiated into epicardial cells.

FIG. 2: Enhanced fibro-fatty gene expression in PKP2 c.2013delC hiPSC-epicardial cells. (A) Volcano plot of RNAseq data showing differentially expressed genes in in PKP2 c.2013delC versus reverted hiPSC-epicardial cells (n=4). (B-C) ClueGO pathway analysis of (B) downregulated and (C) upregulated genes on RNAseq. (D) qPCR, flow cytometry and western blot analysis of WT1 expression in PKP2 c.2013delC and reverted day 80 hiPSC-epicardial cells. (E) qPCR analysis of the indicated fat and fibroblast markers in PKP2 c.2013delC and reverted hiPSC-epicardial cells. Samples were relatively compared to day 0 c.2013delC hiPSC-epicardial cells (n=3). (F, G) Immunofluorescent staining of WT1, PPARG and ACTA2 in PKP2 c.2013delC and reverted hiPSC-epicardial cells. (H) Oil red-O and hematoxylin staining PKP2 c.2013delC and reverted hiPSC-epicardial cells. Data are represented as mean±SEM. *P<0.05, **P<0.01, ***P<0.001, ****P<0.0001. A Student's t-test was used for statistical analysis.

FIG. 3: AP2 factors drive fibro-fatty signaling in mutant hiPSC-epicardial cells. (A) qPCR analysis of AP2A, AP2B and AP2C in hiPSC-epicardial cells at different time points. Samples were relatively compared to day 0 PKP2 c.2013delC hiPSC-epicardial cells (n=3). (B) Immunofluorescent staining of AP2A and AP2C in day 80 PKP2 c.2013delC and reverted hiPSC-epicardial cells. (C-E) qPCR analysis of the indicated genes following siRNA-mediated knock down of (C) AP2A, AP2B and AP2C in PKP2 c.2013delC hiPSC-epicardial cells (n=3) (D) PKP2 in healthy iPSC-epicardial cells (n=6) (E) PKP2 in healthy hiPSC-cardiomyocytes (n=3). Values were relatively compared to si-scramble-treated samples indicated by the dashed lines. *P<0.05, **P<0.01, ***P<0.001, ****P<0.0001, ND=not detected. A Student's t-test was used for statistical analysis.

FIG. 4. JUP knock down induces AP2 and fibro-fatty gene expression. qPCR analysis of the indicated genes following siRNA-mediated knock down of JUP in healthy iPSC-epicardial cells (n=6). Values were relatively compared to si-scramble-treated samples indicated by the dashed lines. *P<0.05, **P<0.01, ***P<0.001, ****P<0.0001, ND=not detected. A Student's t-test was used for statistical analysis.

FIG. 5. Single cell RNA sequencing (scRNAseq) reveals TFAP2A-expressing clusters in PKP2 c.2013delC hiPSC-epicardial cells. (A) Schematic of the scRNAseq protocol. Day 70 PKP2 c.2013delC and reverted hiPSC-epicardial cells were dissociated and sorted into single cells for subsequent sequencing and in silico analysis. (B) Bioanalyzer plots of sorted cells showing intact RNA. (C-D) Monocle 2-generated tSNE maps of scRNAseq displaying 5 distinct cellular clusters in (D) and the origin of each cellular. (E) tSNE maps showing expression levels of different epicardial, fibroblast and fat markers among cellular clutsers. (F-G) Pseudotime trajectory-reconstruction of the different cellular clusters. (H-I) TFAP2A expression presented in (I) pseudotime and (J) tSNE plot.

FIG. 6. TFAP2A mediates epicardial to fibro-fatty signaling through EMT. (A) Western blot analysis of TFAP2A protein levels in PKP2 reverted, PKP2 c.2013delC and TFAP2A siRNA-treated PKP2 c.2013delC hiPSC-epicardial cells. (B) qPCR analysis of the indicated genes following siRNA-mediated knock down of TFAP2A in PKP2 c.2013delC hiPSC-epicardial cells (n=5). Values were relatively compared to si-scramble-treated samples indicated by the dashed lines using Student's t test. (C) qPCR analysis of CDH1/CDH2 expression ratio in the culture timeline and of PKP2 c.2013delC and reverted hiPSC-epicardial cells. (D) Control hiPSC-epicardial cells were triggered to undergo EMT and subsequently transfected with TFAP2A-targeting siRNAs. qPCR analysis of the indicated genes after transfection are shown below. (*P<0.05, **P<0.01, ***P<0.001, ****P<0.0001).

FIG. 7. Cardiac explants from ACM patients show epicardial activation and TFAP2A induction. Upper images in each panel show Masson Trichome stainings of healthy and diseased hearts depicting myocardium in red, fibrosis in blue and adipose tissue in white. Zoomed in images below were stained with WT1 or TFAP2A. Epi indicates epicardium and Myo indicates myocardium. Scale bars in each panel: Upper=10 mm, middle=1 mm, lower=50 um.

### DESCRIPTION OF THE SEQUENCES

TABLE 1
Sequences

| SEQ ID | |
|---|---|
| NO: | Name |
| 1 | TFAP2A variant 1 (Nucleotides) |
| 2 | TFAP2A variant 2 (Nucleotides) |
| 3 | TFAP2A variant 3 (Nucleotides) |
| 4 | TFAP2B (Nucleotides) |
| 5 | TFAP2C (Nucleotides) |
| 6 | TFAP2D (Nucleotides) |
| 7 | TFAP2E (Nucleotides) |
| 8 | Guide 1 |
| 9 | Guide 2 |
| 10 | Repair template sequence |
| 45 | TFAP2A variant 1 (Amino acid) |
| 46 | TFAP2A variant 2 (Amino acid) |
| 47 | TFAP2A variant 3 (Amino acid) |
| 48 | TFAP2B (Amino acid) |
| 49 | TFAP2C (Amino acid) |
| 50 | TFAP2D (Amino acid) |
| 51 | TFAP2E (Amino acid) |

### Examples

### Example 1

### Material and Methods

### CRISPR-Cas9 Targeting

PKP2 mutant (c.2013deIC) iPSCs were kindly provided by Huei-Sheng Vincent Chen. c.2013delC mutation was reverted into wild type (introducing C nucleotide) using CRISPR-Cas9. Two guide RNAs were used to target the PKP2 genomic locus (Guide 1: ATACCAGGACGTGCCGATGC (SEQ ID NO: 8), Guide 2: CCTCCGGCATCGGCACGTCC (SEQ ID NO:9) and a repair template was used to revert the deletion. A mutation in the PAM sequence was included to prevent re-cutting by the guide RNA. Repair template sequence:

(SEQ ID NO: 10)
5′ACACTTTTGGCGATCAAGGACAGATACATCCTTATAACAATTGAATGC
CACAGCCACTCCACGCCCTTGGGGTTGCTCTTTTCCTCGGGCATCGGCAC
GTCCTGGTATTGCTGACCACACACAAAAG3′

### Cell Culture

Human iPSCs were maintained in Essential 8 Medium (Thermofisher Scientific, #A1517001) on Geltrex (Thermofisher Scientific, A1413302)-coated plates. iPSC-epicardial cell differentiation protocol was adapted from Bao et al. 2016.

### Immunofluorescence

Cells were fixed with 4% PFA, blocked with goat serum and incubated for 1 hour with the primary antibodies WT1 (abcam, ab89901), K18 (Life technologies, MS-142-PO), TBX18 (Sigma-Aldrich SAB1412362), ZO1 (Life technologies, 40-2200), PPARG (Santa-cruz sc-7273), ACTA2 (Sigma-Aldrich A5228), AP2A (abcam, ab52222), AP2C (santa-cruz, sc-12762). Cells were subsequently stained with the corresponding Alexa Fluor antibodies (Invitrogen) for 1 hour. Images were taken using the Leica TCS SPE confocal microscope.

### Western Blot

Proteins were isolated from cells using RIPA buffer (50 mM Tris-HCl pH 7.5, 150 mM NaCl, 0.1% SDS, 0.5% sodium deoxycholate (Sigma Aldrich), 1% Triton X-100 (Sigma), protease inhibitor (Roche)) and protein concentration was determined using Bradford assay (BioRad). SDS-PAGE and Western Blot were performed using Mini-PROTEAN Tetra Vertical Electrophoresis Cell with Mini Trans-Blot (Bio-Rad). Membranes were blocked in 3% BSA and subsequently incubated with the primary antibodies WT1 (abcam, ab89901) and GAPDH (Millipore, MAB374). Blots were incubated with Peroxidase-conjugated AffiniPure Rabbit Anti-mouse IgG (IR 315-035-003) and Goat Anti-rabbit IgG secondary antibodies (IR 111-035-003) for 45 min and proteins were visualized using ECL solution (BioRad, #170-5061) on the LAS4000 software program. Western bots were quantified by ImageJ.

### Oil Red O Staining

Cells were stained using the Lipid Oil red 0 staining kit (Sigma, MAK194) according to the manufacturer's instructions.

### Flow Cytometry

Cells were fixed with 70% ethanol and incubated in blocking buffer (PBS, 5% FBS, 1% BSA, 0,5% Triton X-100). Cells were subsequently incubated with the primary antibody WT1 (abcam, ab89901) and the secondary antibody Alexa Fluor 488 donkey anti-rabbit IgG (Thermo Fisher, A21206). WT1+cells were quantified using FACS Aria SORP (BD bioscience).

RNA Isolation and qPCR

Total RNA was isolated using the RNeasy Mini Kit (Qiagen, #74104) according to the manufacturer's instructions. RNA was reverse transcribed into cDNA using iScript cDNA Synthesis Kit (Bio-Rad, #1708891) and used for qPCR using iQ SYBR Green Supermix (Bio-Rad, #170-8885). Transcript levels were normalized for endogenous loading. Primer sequences used are provided in the table 2 below.

TABLE 2
qPCR primers

| SEQ | | | SEQ | |
|---|---|---|---|---|
| ID NO | Gene name | Forward primer | ID NO | Reverse primer |
| 11 | TFAP2A | CTCGATCCACTCCTTACCTCAC | 12 | ATTGCTGTTGGACTTGGACAG |
| 13 | TFAP2B | TAACAGCGGCATGAATCTATTG | 14 | CAGGAAGCCGTCTTTATTCATC |
| 15 | TFAP2C | GATCAGACAGTCATTCGCAAAG | 16 | GTAGAGCTGAGGAGCGACAATC |
| 17 | PKP2 | TGCTAAAGGCTGGCACAA | 18 | TAATCGCTGTGCGTGTAGTG |
| 19 | WT1 | CATGACCTGGAATCAGATGAAC | 20 | CGTGCGTGTGTATTCTGTATTG |
| 21 | UCP1 | AGTACAAAAGTGTGCCCAACTG | 22 | TCGTTTCAGTTGTTCAAAGCAC |
| 23 | PPARG | GTACTGTCGGTTTCAGAAATGC | 24 | ATTCAGCTGGTCGATATCACTG |
| 25 | PPARGC1A | GGTGCAGTGACCAATCAGAA | 26 | AATCCGTCTTCATCCACAGG |
| 27 | COL2A1 | CCTGGTGTCATGGGTTTCC | 28 | GTCCTGCAGCACCTGTCTC |
| 29 | POSTN | TGCCCTTCAACAGATTTTGG | 30 | GCAGCCTTTCATTCCTTCC |
| 31 | FN1 | TCTCATTCAACAAGAAACCACTG | 32 | TTCACGTCTGTCACTTCCACA |
| 33 | CEBPA | ACGATCAGTCCATCCCAGAG | 34 | TTCACATTGCACAAGGCACT |
| 35 | EBF3 | AACAGAGCAAGATCTGTATGTTCG | 36 | CCACAACTTTTCTTGTCACAGC |
| 37 | RORA | CTTTCCCTACTGTTCGTTCACC | 38 | ACGTTATCTGCTGGAGCTCTTC |
| 39 | ACTA2 | CCAGCCATGTATGTGGCTATC | 40 | CCTCATAGATGGGGACATTGTG |
| 41 | COL1A2 | TGATGGAAAAGGAGTTGGACTT | 42 | CAGGTCCTTGGAAACCTTGA |
| 43 | DLK1 | CACGGACTCTGTGGAGAACC | 44 | GCATTCATAGAGGCCATCGT |

siRNA Transfection

siRNA trilencers were purchased from Origene to target TFAP2A (SR304787), TFAP2B (SR304788), TFAP2C (SR304789), PKP2 (SR303544) and JUP (SR302502). 10 nM siRNA trilencer was used for transfection using Lipofectamine 3000 (Life Technologies, L3000008) according to the manufacturer's instructions.

### Results

Generation of PKP2 c.2013deIC and Reverted hiPSC-Epicardial Cells.

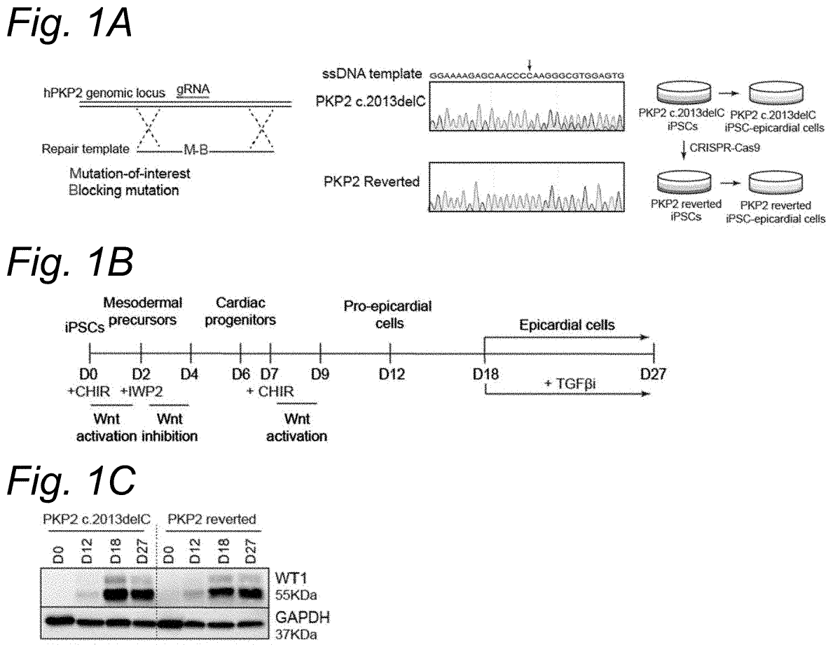

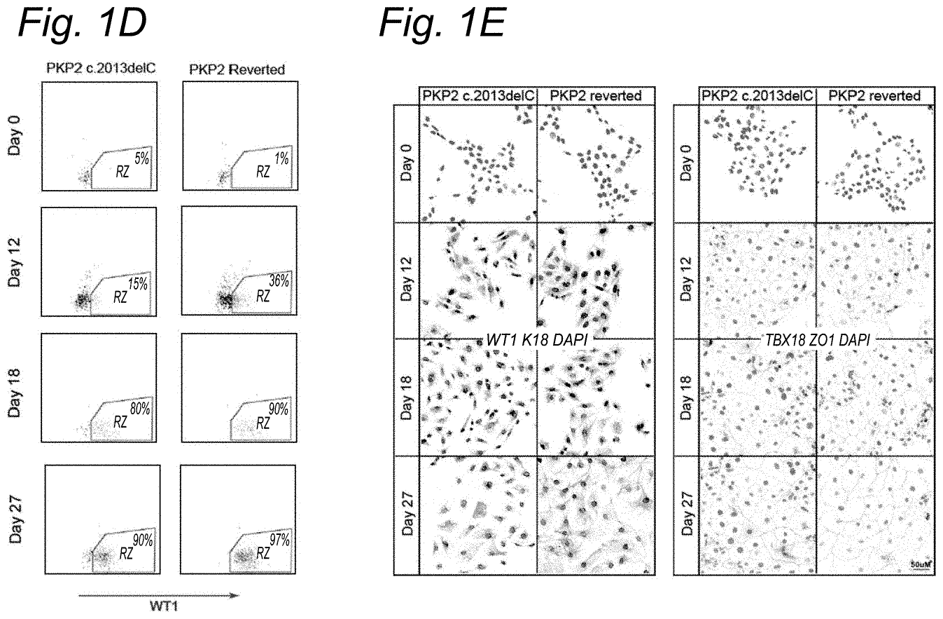

We made use of CRISPR-Cas9 to repair the PKP2 c.2013delC mutation in human induced pluripotent stem cells (hiPSCs) derived from a patient diagnosed with arrhythmogenic cardiomyopathy (ACM) (FIG. 1a). PKP2 c.2013delC and reverted hiPSCs were subsequently differentiated into epicardial cells (hiPSC-epicardial cells) using WNT pathway modulators. (FIG. 1b). This was confirmed by the time-dependent enhanced expression of the epicardial markers WT1, K18, TBX18 and ZO1 (FIG. 1c-e).

PKP2 c.2013delC hiPSC-Epicardial Cells Display Enhanced Fibro-Fatty Gene Signaling.

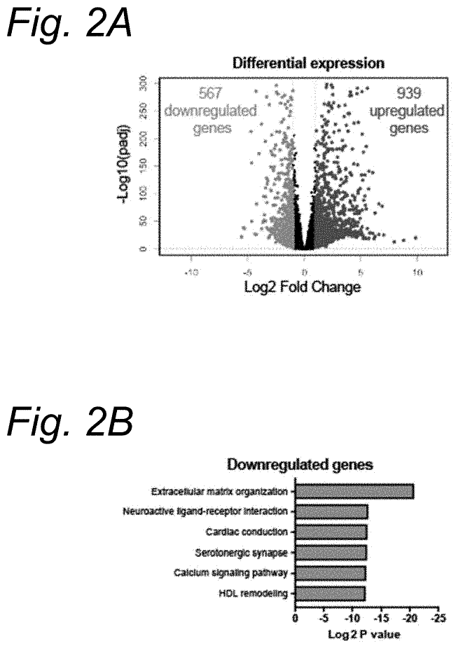

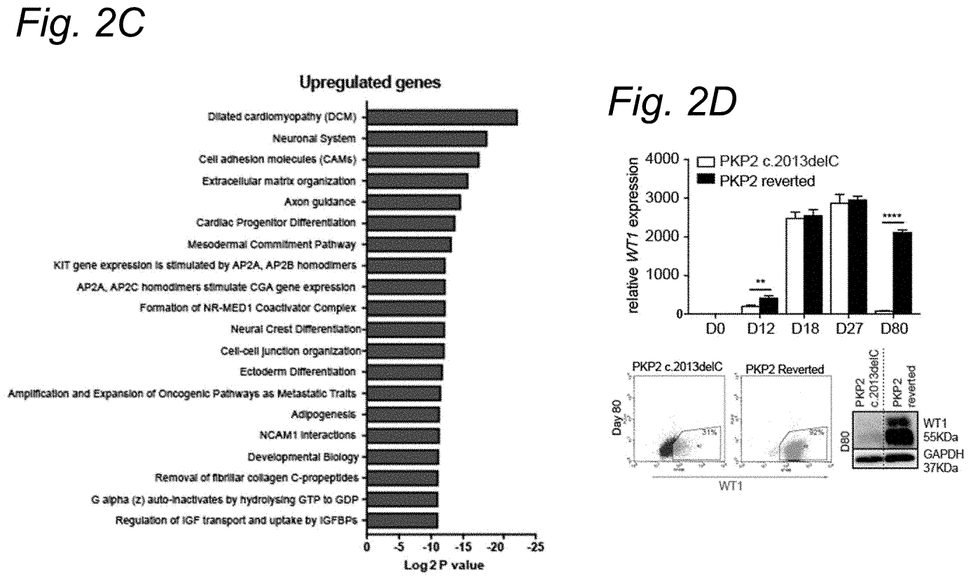

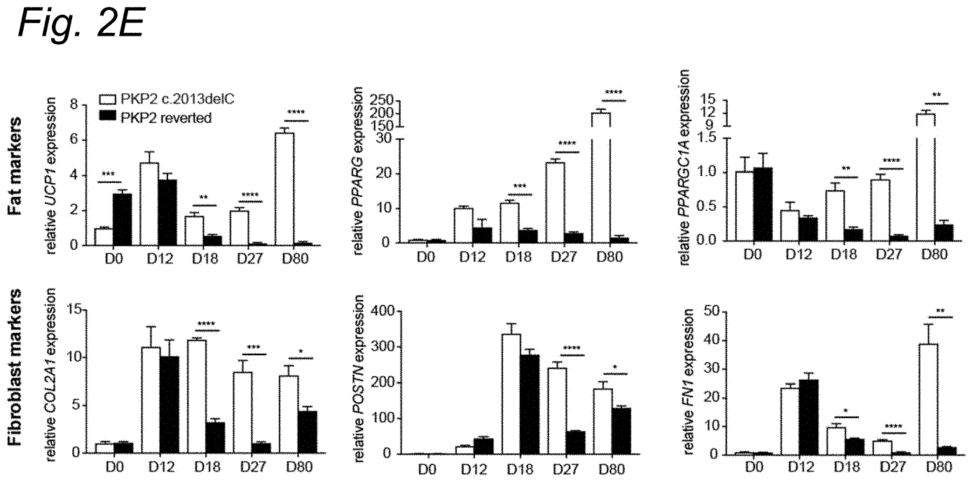

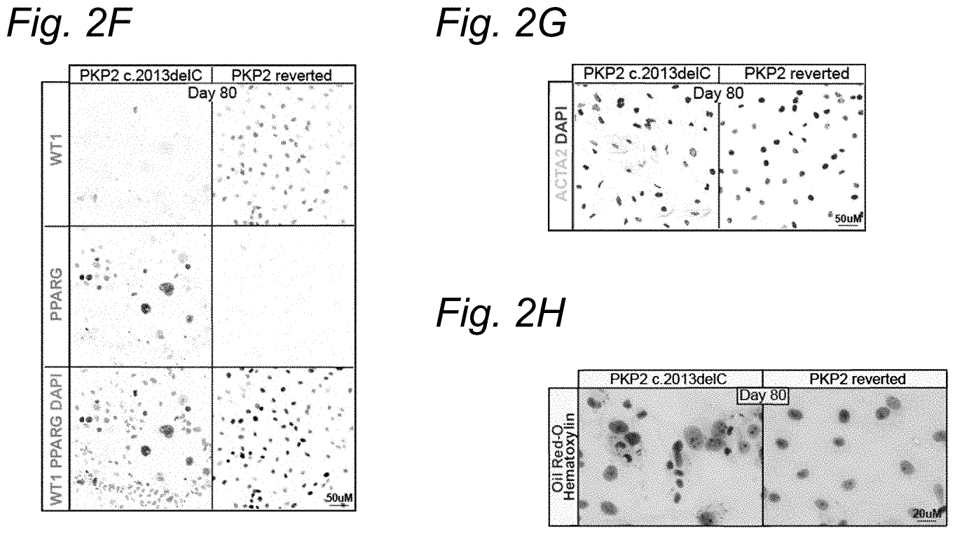

In order to identify the molecular differences between the 2 lines, day 27 PKP2 c.2013delC and reverted hiPSC-epicardial cells were subjected to RNAseq. We identified 567 downregulated (less than −Log 2 fold change) and 939 upregulated (more than Log 2 fold change) genes (FIG. 2a). Gene ontology analysis showed that upregulated genes were mainly involved in cardiomyopathies, cell adhesion pathways and interestingly adipogenesis (FIG. 2c). Having identified molecular differences in the 2 lines despite of their phenotypical similarities, we subjected those cells to long term culturing. When analyzed, day 80 PKP2 c.2013delC, and not the reverted cells, showed a dramatic loss of the expression of the epicardial marker WT1 (FIG. 2d). Those cells profoundly expressed higher levels of the major fat markers PPARG, PPARGC1A and UCP1 as compared to their reverted isogenic controls. A similar expression pattern was also observed in fibroblast markers such as COL2A1, POSTN, FN1 and ACTA2 (FIG. 2e, f). In addition, only PKP2 c.2013delC hiPSC-epicardial cells progressively accumulated lipid droplets as revealed by Oil Red O staining (FIG. 2f). These data indicate that the PKP2 c.2013delC mutation potentially drives a fibro-fatty transition in epicardial cells.

hiPSC-Epicardial Cells Transition into Fibro-Fatty Cells in an AP2-Dependent Mechanism.

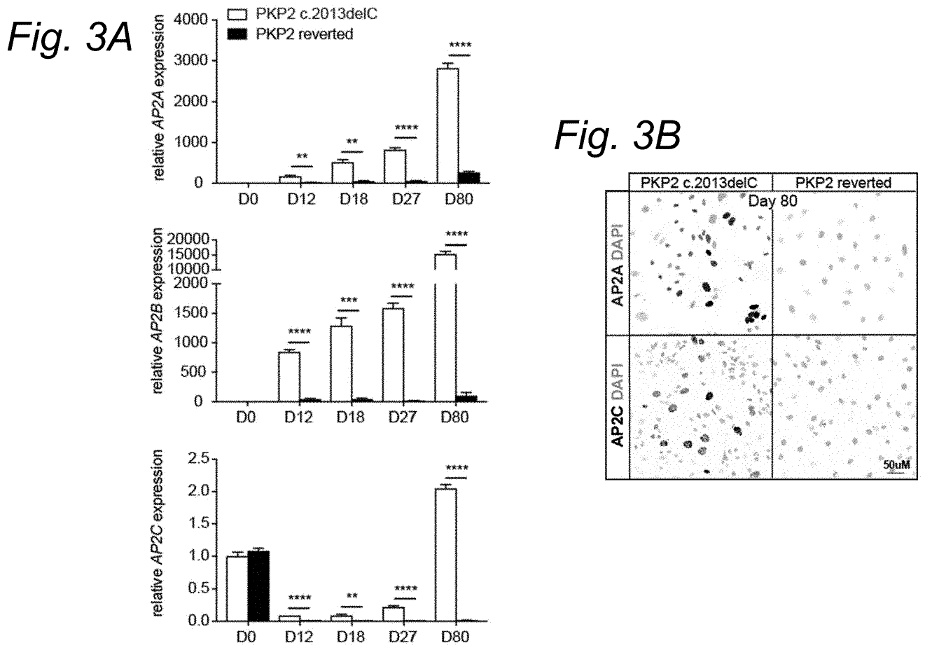

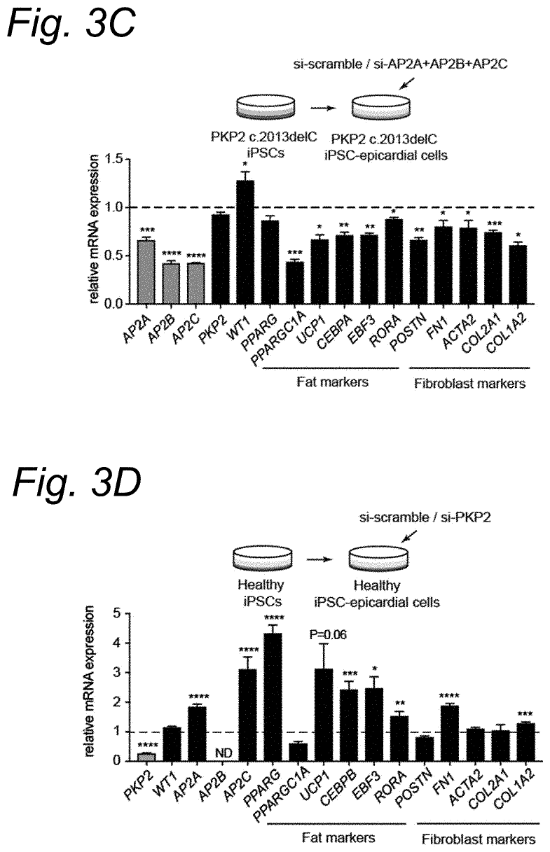

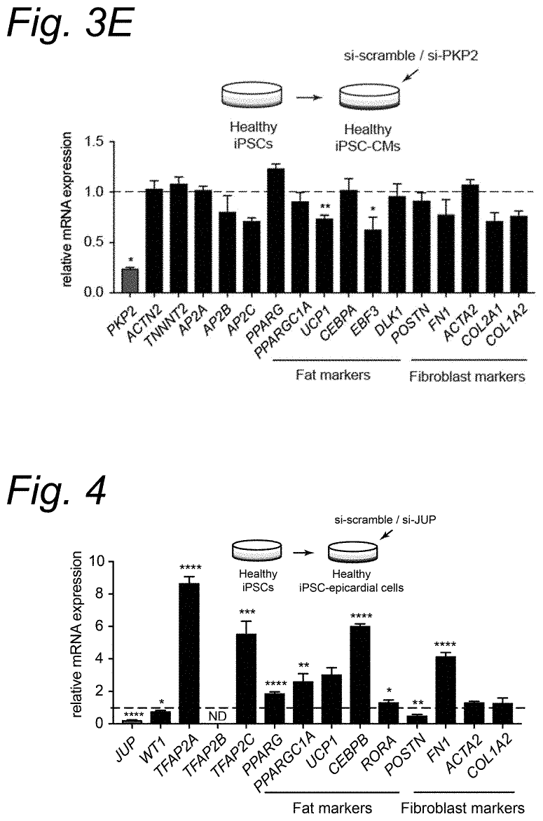

In order to identify the mechanism behind this process, we further analyzed our RNAseq data, by which we identified the Activating Enhancer Binding Protein 2 (AP2) family of transcription factors to be highly induced in PKP2 c.2013delC hiPSC-epicardial cells (FIG. 2c). AP2 factors have been previously identified as master regulators of adipogenesis which indicated their potential in mediating this effect in mutant epicardial cells. Expression of the AP2 factors was further validated by qPCR and immunofluorescent staining (FIG. 3a, b). To investigate whether AP2 factors drive this adipogenic signaling, we set out to use a mixture of targeting siRNAs to knock down AP2A, AP2B and AP2C in PKP2 c.2013delC hiPSC-epicardial cells. After 48 h of transfection, we could detect a significant reduction in AP2 expression as well as in fat and fibroblast markers suggesting an AP2-mediated fibro-fatty signaling in epicardial cells (FIG. 3c). The PKP2-dependence of these findings was further validated in healthy hiPSC-derived epicardial cells treated with siRNAs targeting PKP2, which recapitulated the observations made in the mutant cells (FIG. 3d). To identify whether PKP2 knock down in other cardiac cells would trigger the same effect, we differentiated cardiomyocytes from healthy hiPSCs and performed the same PKP2 knock down experiment. We did not observe any changes in fibro-fatty gene expression in those cells indicating that this effect is epicardial cells-specific (FIG. 3e). To validate whether the induction of AP2 factors and fibo-fatty genes is triggered by a dysregulation of other desmosomal proteins, we knocked down JUP in healthy hiPSC-derived epicardial cells. We observed a significant increase of AP2 and fibro-fatty genes in si-JUP treated samples (FIG. 4) indicating that this is a common mechanism in ACM.

### CONCLUSION

We generated epicardial cells from induced pluripotent stem cells (iPSCs) of an ACM patient carrying a mutation in the desmosomal gene Plakophilin-2 (PKP2) (c.2013delC) as well as their isogenic control in which the mutation was reverted into wild type using CRISPR-Cas9.

Both lines exhibited a similar epicardial state when cultured up to 1 month as assessed by qPCR, western blotting, flow cytometry and immunofluorescent staining of epicardial markers. However, when subjected to RNA sequencing, only the mutant line highly expressed genes enriched in adipogenic signaling, which suggested the potential of those cells towards adipocytic differentiation. Indeed, when subjected to long term culturing conditions, mutant PKP2 cells and not their isogenic controls displayed a high expression of adipogenic and fibroblast markers and accumulated lipid droplets as seen by Oil-Red-O staining.

Follow up experiments showed that AP2 family of genes showed an increase in the PKP2 mutant cells. AP2 genes are known for their roles in in development, cell growth and differentiation, but have not been studied in the heart. Interestingly, a recent study identified those factors to act as regulators of lipid droplet biogenesis.

To test whether AP2 might be regulating the fibro-fatty phenotype seen in the PKP2 mutant epicardial cells, using siRNAs against AP2 we observed a reduced expression of fibroblast and lipid markers and an increased expression of epicardial markers in the siRNA-transfected cells, indicating that PKP2 mutant epicardial cells are undergoing differentiation into fibroblasts and adipocytes in an AP2-dependent mechanism.

To show whether the induction of AP2 is a direct effect of a decreased level of PKP2 in the patient cells we used an siRNA against PKP2 in healthy epicardial cells. In doing so we were able to show that PKP2 inhibition leads to an induction in AP2 and fibro-fatty markers.

### Example 2

TFAP2A Mediates Fibro-Fatty Transition in PKP2 c.2013deIC hiPSC-Epicardial Cells.

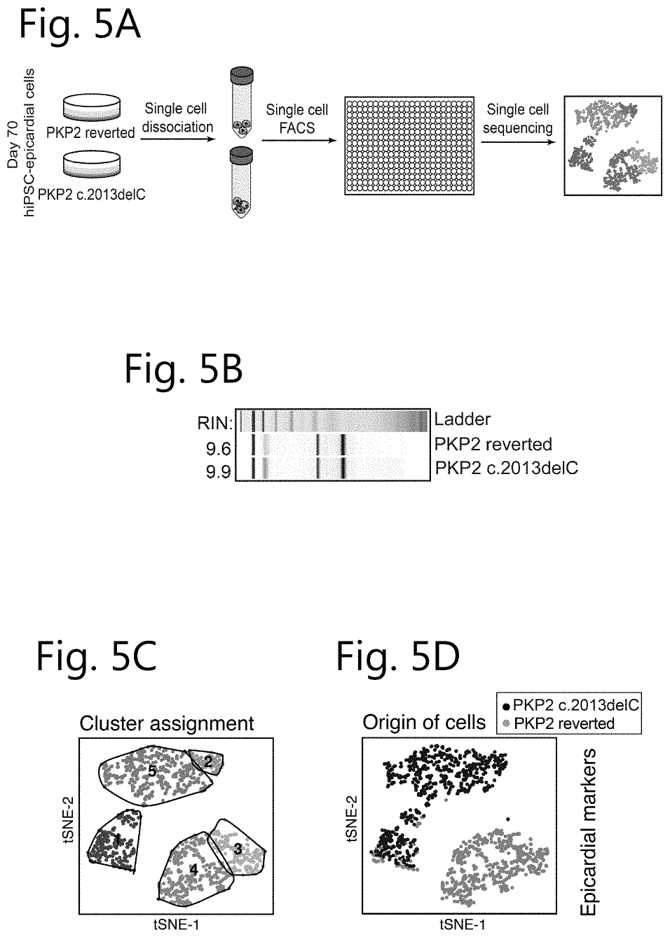

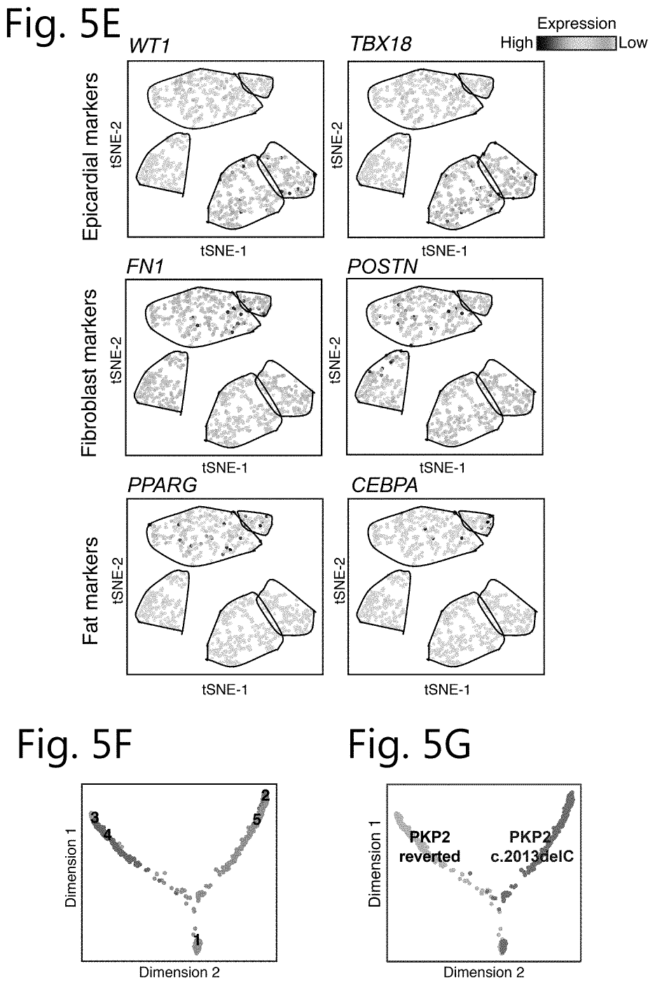

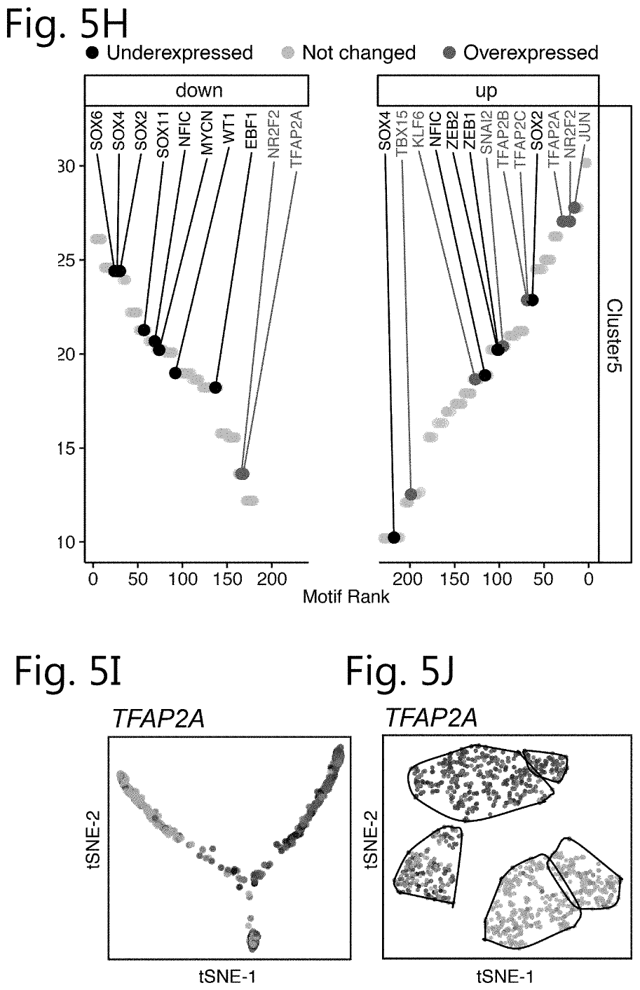

In order to gain more insights into the subcellular identities generated in long term cultures of PKP2 c.2013delC and reverted hiPSC-epicardial cells, we subjected day 70 cultures to single cell RNA sequencing (scRNA-seq) (FIG. 5A). After sorting, we obtained >96% live cells and intact RNA as checked on Bioanalyzer (FIG. 5B). Single cell RNA sequencing analysis showed that all cells clustered into 5 distinct cellular clusters as shown in the representative t-Distributed Stochastic Neighbor Embedding (t-SNE) maps (FIG. 5C-D). The differential expression of epicardial, fibroblast and fat markers between the 2 lines was further validated between cellular clusters indicating an epicardial to fibro-fatty switch in the mutant cells (FIG. 5E). We then used Monocle's pseudotime trajectory-reconstruction algorithm to create a branched tree structure representing directions of cellular differentiation (FIG. 5F-G). This analysis could give an indication of the cellular events triggering the epicardial to fibro-fatty cell transition. We generated a list of the top 10 transcription factors which showed a significant differential expression between the branches, among which Transcription factor TFAP2A showed a clear induction in the mutant clusters, suggesting a potential role in driving this phenotype early during differentiation (FIG. 51, J). Furthermore, we used HOMER (Hypergeometric Optimization of Motif EnRichment) to identify transcription factors with enriched sequence motifs in differentially expressed genes between the two lines. In doing so, we observed a significant enrichment for motifs recognized by TFAP2A in cluster 5 which mainly contains mutant cells (FIG. 5H). Altogether, these data further suggested a role for TFAP2A in fibro-fatty signaling in epicardial cells.

### TFAP2A Mediates Epicardial to Fibro-Fatty Transition Through EMT.

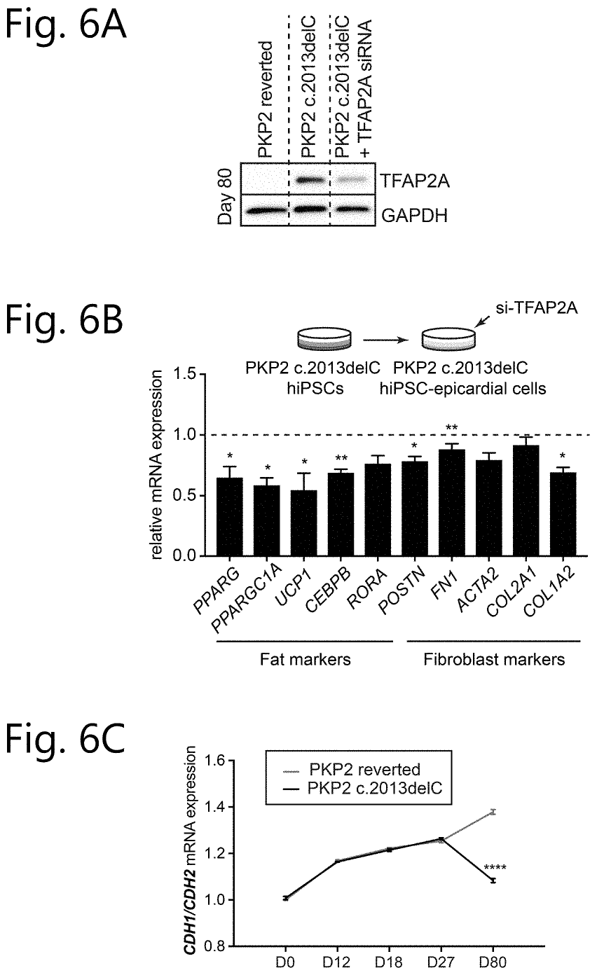

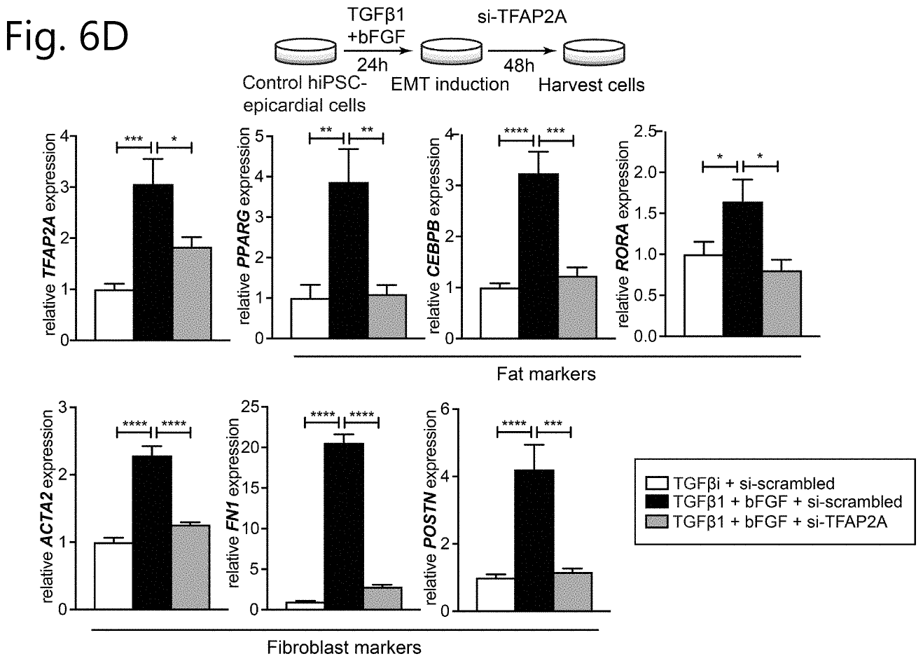

To investigate whether TFAP2A is sufficient to induce to this fibro-fatty gene signaling, we set out to use a mixture of targeting siRNAs (as described here above) to knock down TFAP2A in PKP2 c.2013delC hiPSC-epicardial cells. After 48 h of transfection, we could detect a significant reduction in TFAP2A expression (FIG. 6A) as well as in several fibroblast and fat markers suggesting a potential TFAP2A-mediated fibro-fatty signaling in epicardial cells (FIG. 6B). Epicardial cells reside in an epithelial state which upon activation can undergo epithelial-to-mesenchymal transition (EMT) and start cellular differentiation. Therefore, we postulated that desmosomal suppression might act as an EMT-driving trigger. We analyzed the expression ratio of E-cadherin versus N-cadherin (CDH1/CDH2) in PKP2 c.2013delC hiPSC-epicardial cells versus the reverted controls, which is a measure of the epithelial versus mesenchymal state of the cells. We observed a similar expression ratio among our culture timeline up to 1 month. However, by day 80 only mutant cells displayed a dramatic reduction in this ratio indicating an EMT switch (FIG. 6C). To investigate whether artificially-induced EMT would promote a similar fibro-fatty phenotype, we cultured control hiPSC-epicardial cells in the presence of TGFβ1 and bFGF, well known inducers of epicardial EMT. As expected, TGFβ1+bFGF-treated cells showed a significant induction of TFAP2A as well as fibroblast, smooth muscle and fat cell markers. However, addition of siRNAs targeting TFAP2A abolished this effect (FIG. 6D). These results suggest that loss of desmosomal proteins can affect the epicardial integrity leading to its activation, EMT and cellular differentiation in an TFAP2A-dependent mechanism.

### Human Explanted ACM Hearts Express WT1 and TFAP2A

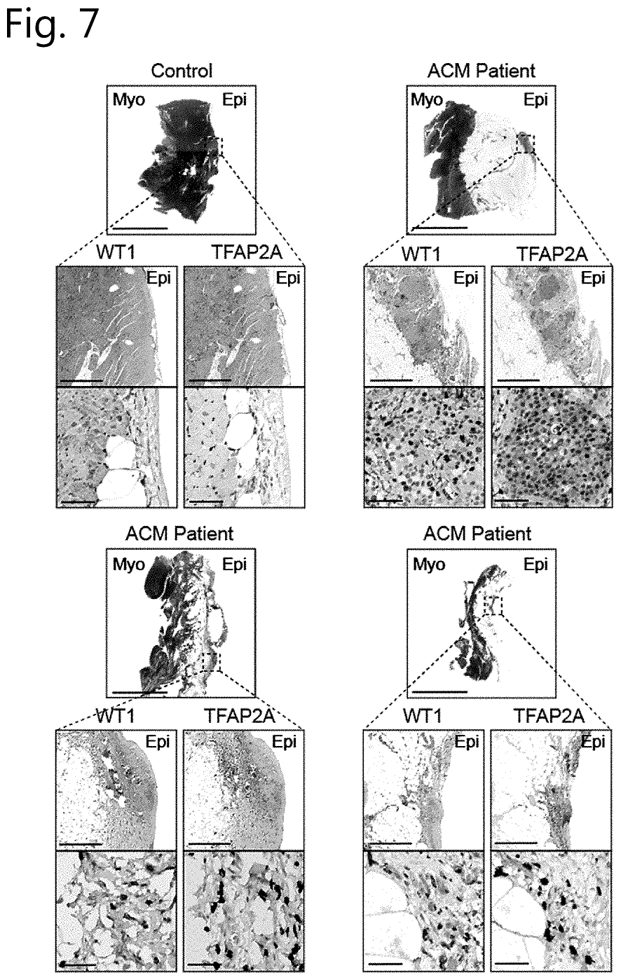

In line with our observations in the in vitro hiPSC-epicardial cell cultures, we performed immunohistological stainings on heart sections explanted from healthy and ACM patients. In the healthy heart, the epicardium resided as a thin membranous layer surrounding the myocardium with very little fat and fibrosis underneath. However, hearts of ACM patients displayed a thicker sub-epicardium covering massive fibro-fatty infiltrates. In addition, those epicardial/subepicardial cells expressed WT1, a phenomenon also observed in animal models after cardiac injury, further suggesting epicardial activation in diseased hearts. WT1 induction was associated with TFAP2A expression in the sub-epicardial areas and among fibro-fatty infiltrates further supporting our findings revealing TFAP2A as a key factor in epicardial EMT and fibro-fatty tissue transition (FIG. 7).

### REFERENCES

Gemayel C, Pelliccia A, Thompson P D, Arrhythmogenic right ventricular cardiomyopathy. J Am Coll Cardiol. 2001 December; 38(7):1773-81
Saffitz J E. The pathobiology of arrhythmogenic cardiomyopathy. Annual Review of Pathology: Mechanisms of Disease. 2011 Feb. 28; 6:299-321.
Gorman, L., Suter, D., Emerick, V., Schumperli, D., and Kole, R. (1998). Stable alteration of pre-mRNA splicing patterns by modified U7 small nuclear RNAs. Proceedings of the National Academy of Sciences of the United States of America 95, 4929-4934
Scott C C, Vossio S, Rougemont J, Gruenberg J. TFAP2 transcription factors are regulators of lipid droplet biogenesis. eLife. 2018 Sep. 26; 7:e36330.
Bao X, Lian X, Hacker T A, Schmuck E G, Qian T, Bhute V J, Han T, Shi M, Drowley L, Plowright A, Wang Q D, Goumans M J, Palecek S P. Long-term self-renewing human epicardial cells generated from pluripotent stem cells under defined xeno-free conditions. Nat Biomed Eng. 2016; 1. pii: 0003.
Corrado D, Basso C, Judge D P. Arrhythmogenic Cardiomyopathy. Circulation Research. 2017 Sep. 15; 121(7):784-802.

1.-15. (canceled)

16. A method for treating or preventing cardiac disease wherein fibro-fatty replacement is part of the disease etiology, wherein the method comprises administering to a subject in need thereof at least one of:
a) an agent that causes a reduction in expression in at least one TFAP2 subtype; and,
b) an agent that causes a reduction in TFAP2-induced transcription.

17. The method according to claim 16, wherein the agent is at least one of:
a) an inhibitor of TFAP2; and
b) an agent that causes an increase in expression of PKP2.

18. The method according to claim 16, wherein the agent is (a source of) at least one of a genome editing complex, an antibody, a compound, preferably the agent is a nucleic acid molecule, a siRNA, miRNA, more preferably, the agent is at least one of: a genome editing complex that restores PKP2 deficiency or inactivates TFAP2, a neutralizing antibody against TFAP2 and an siRNA complementary to TFAP2 mRNA.

19. The method according to claim 17, wherein the agent that causes an increase in expression of PKP2 is an inhibitor of Wnt3a, preferably the inhibitor of Wnt3a is selected from an anti-Wnt3a antibody, Tricostatin A, hexachlorophene and niclosamide.

20. The method according to claim 16, wherein the agent is administered to a subject intermittently or continuously.

21. The method according to claim 16, wherein the agent is administered locally to the epicardium/pericardial sac region.

22. The method according to claim 16, wherein TFAP2 expression is reduced by at least 10%, 20%, 30%, 40%, 60%, 80%, or more or wherein TFAP2-induced transcription is reduced by at least 10%, 20%, 30%, 40%, 60%, 80%, or more, preferably the reduction of the reduction of TFAP2 expression or the reduction of TFAP2-induced transcription is determined by PCR or immunostaining.

23. The method according to claim 16, wherein the cardiac disease is at least one of arrhythmogenic cardiomyopathy, atrial fibrillation, myocardial infarction and dilated cardiomyopathy.

24. The method according to claim 16, wherein the cardiac disease is caused by a mutation in a desmosomal protein, preferably wherein the desmosomal protein is plakophilin-2 (PKP2), more preferably wherein the protein is PKP2 and the mutation is c.2013delC.

25. An in vivo, in vitro, or ex vivo method for reducing TFAP2 expression, the method comprising the step of contacting a cell with:
a) an agent that causes a reduction in expression in at least one TFAP2 subtype; or,
b) an agent that causes a reduction in TFAP2-induced transcription

26. A method for identifying an agent that causes at least one of a reduction in TFAP2 expression, and a reduction in TFAP2-induced transcription, the method comprising the steps of:
a) contacting a PKP2-deficient epicardial cell with a candidate agent;
b) determining in the cell in a) at least one of the level of TFAP2 expression and the level of TFAP2-induced transcription;
c) identifying the agent as an agent that causes a reduction in TFAP2 expression if the level of TFAP2 expression as in determined in b) is less that the level in a corresponding control cell in the absence of the agent, and,
d) identifying the agent as an agent that causes a reduction in TFAP2-induced transcription if the level of TFAP2 expression as in determined in b) is less that the level in a corresponding control cell in the absence of the agent.
